(19) United States
(12) Patent Application Publication
(10) Pub. No.: US 2026/0159610 A1
(43) Pub. Date: Jun. 11, 2026

(54) ANTIBODIES AND COMPOSITIONS FOR USE IN DETECTING OR CAPTURING A POLYPEPTIDE IN A SAMPLE, AND METHODS FOR DETECTING OR CAPTURING A POLYPEPTIDE IN A SAMPLE

(71) Applicant: CHUGAI SEIYAKU KABUSHIKI KAISHA, Tokyo (JP)

(72) Inventors: Eriko MATSUDA, Kanagawa (JP); Mitsuko SHIBUYA, Shizuoka (JP); Masanobu NISHIDATE, Kanagawa (JP)

(73) Assignee: CHUGAI SEIYAKU KABUSHIKI KAISHA, Tokyo (JP)

(21) Appl. No.: 19/456,668

(22) Filed: Jan. 22, 2026

Related U.S. Application Data
(62) Division of application No. 17/603,684, now Pat. No. 12,559,571.

(30) Foreign Application Priority Data
Apr. 19, 2019 (JP) 2019-079740

(51) Int. Cl. C07K 16/42; C07H 21/00; C07K 16/00

(52) U.S. Cl. CPC C07K 16/4283; C07H 21/00; C07K 16/00; C07K 2317/30; C07K 2317/56; C07K 2317/92

(57) ABSTRACT

Provided herein is an antibody to detect and/or capture a polypeptide comprising a modified IgG heavy chain constant region that is derived from any one of constant regions in human naturally occurring IgGs or that is derived from a chimeric constant region obtained from at least two selected from the constant regions in human naturally occurring IgGs, a composition comprising the antibody for use in detecting or capturing a polypeptide in a sample, and a method for detecting or capturing a polypeptide in a sample using the antibody.

### CROSS-REFERENCE TO RELATED APPLICATIONS

This application is a divisional of U.S. patent application Ser. No. 17/603,684, filed Oct. 14, 2021, which is a U.S. National Phase of PCT Application No. PCT/JP2020/016813, filed Apr. 17, 2020, which claims the benefit of Japanese Patent Application No. 2019-079740, filed Apr. 19, 2019, each of which is incorporated herein by reference in its entirety.

### REFERENCE TO SEQUENCE LISTING SUBMITTED ELECTRONICALLY

The content of the electronically submitted sequence listing (Name: 6663_0389_Sequence_Listing.xml; Size: 152,568 bytes; and Date of Creation: Jan. 16, 2026) is incorporated herein by reference in its entirety.

### TECHNICAL FIELD

The present invention relates to antibodies and compositions for use in detecting or capturing a polypeptide in a sample, and methods for detecting or capturing a polypeptide in a sample.

### BACKGROUND ART

Hybridoma technology has enabled the production of monoclonal antibodies and this monoclonal antibody technology has been widely used in many scientific fields (NPL1). After this technological achievement, further efforts were made in the field of therapeutic and diagnostic antibodies. 30 years have passed since the first US approval of monoclonal antibody therapy (NPL2). More than 30 antibodies have been approved by the FDA and a significant number of candidates are under clinical and pre-clinical evaluation. So far, monoclonal antibodies have remained the standard therapeutic molecules and are used in various disease areas such as cancer, autoimmune diseases, respiratory diseases, infectious diseases, and neural diseases (NPL3).

In order to increase the merit of therapeutic antibodies, many different types of engineered Fc modifications to improve functions such as those for antibody-dependent cell-mediated cytotoxicity enhancement, complement dependent cytotoxicity enhancement, antibody half-life extension, antigen clearance modulation, and facilitation of heavy chain heterodimerization, were identified (NPL4).

Antibodies that specifically bind to engineered Fc regions but not to a wild-type Fc have been reported (NPL5, PTL1). It was proven that antibodies against engineered Fc regions are quite useful for various purposes.

### CITATION LIST

### Patent Literature

[PTL1] WO2017072210A1

### Non Patent Literature

[NPL1] Kohler, G. et al., Nature 256:495-497 (1975)
[NPL2] Reichert, J. M. et al., Curr. Pharm. Biotechnol. 9:423-430 (2008)
[NPL3] Lagasse HAD et al. F1000Research 2017, 6 (F1000 Faculty Rev): 113
[NPL4] Mimoto et al., Curr. Pharm. Biotechnol. 17:1298-1314 (2016)
[NPL5] Yu et al., Antimicrob Agents Chemother. 61 (2016)

### SUMMARY OF INVENTION

We have provided some antibodies comprising a modified IgG heavy chain constant region that is derived from any one of constant regions in human naturally occurring IgGs or that is derived from a chimeric constant region obtained from at least two selected from the constant regions in human naturally occurring IgGs. The antibodies include, for example, satralizumab, nemolizumab, emicizumab, SKY59 (Crovalimab), AMY109, and GYM329. For example, the modified IgG heavy chain constant region in one of them comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438 and Glu at position 440 (all positions according to the EU numbering system). The present invention provides antibodies that specifically bind to, detect, and/or capture a polypeptide comprising the modified IgG heavy chain constant region or an epitope in it, compositions comprising the antibody, and methods of using the antibody.

Specifically, the present invention relates to [1] to [25] below.

[1] An isolated antibody which specifically binds to a modified IgG heavy chain constant region that is derived from any one of constant regions in human naturally occurring IgGs or that is derived from a chimeric constant region obtained from at least two selected from the constant regions in human naturally occurring IgGs, wherein the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

[2] The antibody of [1], wherein the antibody substantially does not bind to said any one of constant regions in human naturally occurring IgGs and the chimeric constant region obtained from at least two selected from the constant regions in human naturally occurring IgGs.

[3] The antibody of [1] or [2], wherein the constant regions in human naturally occurring IgGs are an IgG1 constant region consisting of the amino acid sequence of SEQ ID NO: 106, an IgG2 constant region consisting of the amino acid sequence of SEQ ID NO: 107, an IgG3 constant region consisting of the amino acid sequence of SEQ ID NO: 108, and an IgG4 constant region consisting of the amino acid sequence of SEQ ID NO: 109.

[4] The antibody of any one of [1] to [3], wherein said modified heavy chain constant region is derived from a chimeric constant region obtained from constant regions in human naturally occurring IgG1 and IgG4.

[5] The antibody of any one of [1] to [4], wherein the modified IgG heavy chain constant region comprises at least one amino acid selected from the group consisting of Arg at position 235, Arg at position 236, Lys at position 239, Leu at position 428, Ala at position 434, Arg at position 438, and Glu at position 440 (all positions according to the EU numbering system).

[6] The antibody of any one of [1] to [5], wherein the modified IgG heavy chain constant region comprises at least one amino acid selected from the group consisting of Arg at position 235, Arg at position 236, and Lys at position 239 (all positions according to the EU numbering system).

[7] The antibody of any one of [1] to [6], wherein the modified IgG heavy chain constant region comprises Arg at position 235, and either or both of Arg at position 236 and Lys at position 239 (all positions according to the EU numbering system).

[8] The antibody of any one of [1] to [7], which binds to the part consisting of the amino acid sequence RRGPK (SEQ ID NO: 104) or RRGPS (SEQ ID NO: 117) in the modified IgG heavy chain constant region.

[9] The antibody of any one of [1] to [8], which comprises any one of following (a) to (f):
(a) variable regions that comprise
   HVR-H1 comprising the amino acid sequence of SEQ ID NO: 33,
   HVR-H2 comprising the amino acid sequence of SEQ ID NO: 45,
   HVR-H3 comprising the amino acid sequence of SEQ ID NO: 57,
   HVR-L1 comprising the amino acid sequence of SEQ ID NO: 69,
   HVR-L2 comprising the amino acid sequence of SEQ ID NO: 81, and
   HVR-L3 comprising the amino acid sequence of SEQ ID NO: 93;
(b) variable regions that comprise
   HVR-H1 comprising the amino acid sequence of SEQ ID NO: 34,
   HVR-H2 comprising the amino acid sequence of SEQ ID NO: 46,
   HVR-H3 comprising the amino acid sequence of SEQ ID NO: 58,
   HVR-L1 comprising the amino acid sequence of SEQ ID NO: 70,
   HVR-L2 comprising the amino acid sequence of SEQ ID NO: 82, and
   HVR-L3 comprising the amino acid sequence of SEQ ID NO: 94;
(c) variable regions that comprise
   HVR-H1 comprising the amino acid sequence of SEQ ID NO: 37,
   HVR-H2 comprising the amino acid sequence of SEQ ID NO: 49,
   HVR-H3 comprising the amino acid sequence of SEQ ID NO: 61,
   HVR-L1 comprising the amino acid sequence of SEQ ID NO: 73,
   HVR-L2 comprising the amino acid sequence of SEQ ID NO: 85, and
   HVR-L3 comprising the amino acid sequence of SEQ ID NO: 97;
(d) variable regions that comprise
   HVR-H1 comprising the amino acid sequence of SEQ ID NO: 38,
   HVR-H2 comprising the amino acid sequence of SEQ ID NO: 50,
   HVR-H3 comprising the amino acid sequence of SEQ ID NO: 62,
   HVR-L1 comprising the amino acid sequence of SEQ ID NO: 74,
   HVR-L2 comprising the amino acid sequence of SEQ ID NO: 86, and
   HVR-L3 comprising the amino acid sequence of SEQ ID NO: 98;
(e) variable regions that comprise
   HVR-H1 comprising the amino acid sequence of SEQ ID NO: 39,
   HVR-H2 comprising the amino acid sequence of SEQ ID NO: 51,
   HVR-H3 comprising the amino acid sequence of SEQ ID NO: 63,
   HVR-L1 comprising the amino acid sequence of SEQ ID NO: 75,
   HVR-L2 comprising the amino acid sequence of SEQ ID NO: 87, and
   HVR-L3 comprising the amino acid sequence of SEQ ID NO: 99; and
(f) variable regions that comprise
   HVR-H1 comprising the amino acid sequence of SEQ ID NO: 41,
   HVR-H2 comprising the amino acid sequence of SEQ ID NO: 53,
   HVR-H3 comprising the amino acid sequence of SEQ ID NO: 65,
   HVR-L1 comprising the amino acid sequence of SEQ ID NO: 77,
   HVR-L2 comprising the amino acid sequence of SEQ ID NO: 89, and
   HVR-L3 comprising the amino acid sequence of SEQ ID NO: 101.

[10] The antibody of any one of [1] to [5], wherein the modified IgG heavy chain constant region comprises at least one selected from the group consisting of Leu at position 428, Ala at position 434, Arg at position 438, and Glu at position 440 (all positions according to the EU numbering system).

[11] The antibody of any one of [1] to [5] and [10], wherein the modified IgG heavy chain constant region comprises Leu at position 428, Ala at position 434, Arg at position 438, and Glu at position 440, and optionally threonine at position 436 (all positions according to the EU numbering system).

[12] The antibody of any one of [1] to [5], [10], and [11], which binds to the part consisting of the amino acid sequence LHEALHAHYTRKE (SEQ ID NO: 105) or LHEALHAHTTRKE (SEQ ID NO: 118) in the modified IgG heavy chain constant region.

[13] The antibody of any one of [1] to [5] and [10] to [12], which comprises any one of following (g) to (l):
(g) variable regions that comprise
   HVR-H1 comprising the amino acid sequence of SEQ ID NO: 32,
   HVR-H2 comprising the amino acid sequence of SEQ ID NO: 44,
   HVR-H3 comprising the amino acid sequence of SEQ ID NO: 56,
   HVR-L1 comprising the amino acid sequence of SEQ ID NO: 68,
   HVR-L2 comprising the amino acid sequence of SEQ ID NO: 80, and
   HVR-L3 comprising the amino acid sequence of SEQ ID NO: 92;
(h) variable regions that comprise
   HVR-H1 comprising the amino acid sequence of SEQ ID NO: 35,
   HVR-H2 comprising the amino acid sequence of SEQ ID NO: 47,
   HVR-H3 comprising the amino acid sequence of SEQ ID NO: 59,
   HVR-L1 comprising the amino acid sequence of SEQ ID NO: 71,
   HVR-L2 comprising the amino acid sequence of SEQ ID NO: 83, and
   HVR-L3 comprising the amino acid sequence of SEQ ID NO: 95;
(i) variable regions that comprise
   HVR-H1 comprising the amino acid sequence of SEQ ID NO: 36,
   HVR-H2 comprising the amino acid sequence of SEQ ID NO: 48,
   HVR-H3 comprising the amino acid sequence of SEQ ID NO: 60,
   HVR-L1 comprising the amino acid sequence of SEQ ID NO: 72,
   HVR-L2 comprising the amino acid sequence of SEQ ID NO: 84, and
   HVR-L3 comprising the amino acid sequence of SEQ ID NO: 96;
(j) variable regions that comprise
   HVR-H1 comprising the amino acid sequence of SEQ ID NO: 40,
   HVR-H2 comprising the amino acid sequence of SEQ ID NO: 52,
   HVR-H3 comprising the amino acid sequence of SEQ ID NO: 64,
   HVR-L1 comprising the amino acid sequence of SEQ ID NO: 76,
   HVR-L2 comprising the amino acid sequence of SEQ ID NO: 88, and
   HVR-L3 comprising the amino acid sequence of SEQ ID NO: 100;
(k) variable regions that comprise
   HVR-H1 comprising the amino acid sequence of SEQ ID NO: 42,
   HVR-H2 comprising the amino acid sequence of SEQ ID NO: 54,
   HVR-H3 comprising the amino acid sequence of SEQ ID NO: 66,
   HVR-L1 comprising the amino acid sequence of SEQ ID NO: 78,
   HVR-L2 comprising the amino acid sequence of SEQ ID NO: 90, and
   HVR-L3 comprising the amino acid sequence of SEQ ID NO: 102; and
(l) variable regions that comprise
   HVR-H1 comprising the amino acid sequence of SEQ ID NO: 43,
   HVR-H2 comprising the amino acid sequence of SEQ ID NO: 55,
   HVR-H3 comprising the amino acid sequence of SEQ ID NO: 67,
   HVR-L1 comprising the amino acid sequence of SEQ ID NO: 79,
   HVR-L2 comprising the amino acid sequence of SEQ ID NO: 91, and
   HVR-L3 comprising the amino acid sequence of SEQ ID NO: 103.

[14] An isolated antibody which binds to the same epitope as the antibody of any one of [1] to [13].

[15] An isolated antibody which specifically binds to a modified IgG heavy chain constant region, wherein binding of the antibody to the modified IgG heavy chain constant region competes with the antibody of any one of [1] to [14], wherein the modified IgG heavy chain constant region is derived from any one of constant regions in human naturally occurring IgGs or is derived from a chimeric constant region obtained from at least two selected from the constant regions in human naturally occurring IgGs, and comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

[16] A composition for use in detecting or capturing a polypeptide in a sample, wherein the composition comprises the antibody of any one of [1] to [15].

[17] The composition of [16], wherein the polypeptide comprises a modified IgG heavy chain constant region that is derived from any one of constant regions in human naturally occurring IgGs or that is derived from a chimeric constant region obtained from at least two selected from the constant regions in human naturally occurring IgGs, wherein the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

[18] The composition of [16], wherein the polypeptide comprises any one of the amino acid sequence consisting of RRGPK (SEQ ID NO: 104), the amino acid sequence consisting of RRGPS (SEQ ID NO: 117), the amino acid sequence consisting of LHEALHAHYTRKE (SEQ ID NO: 105), and the amino acid sequence consisting of LHEALHAHTTRKE (SEQ ID NO: 118).

[19] The composition of [18], wherein the polypeptide comprises a modified IgG heavy chain constant region that comprises any one of the amino acid sequence consisting of RRGPK (SEQ ID NO: 104), the amino acid sequence consisting of RRGPS (SEQ ID NO: 117), the amino acid sequence consisting of LHEALHAHYTRKE (SEQ ID NO: 105), and the amino acid sequence consisting of LHEALHAHTTRKE (SEQ ID NO: 118).

[20] A method for detecting or capturing a polypeptide in a sample, wherein the method comprises contacting the sample with the antibody of any one of [1] to [15] or with the composition of any one of [16] to [19].

[21] The method of [20], wherein the polypeptide comprises a modified IgG heavy chain constant region that is derived from any one of constant regions in human naturally occurring IgGs or that is derived from a chimeric constant region obtained from at least two selected from the constant regions in human naturally occurring IgGs, wherein the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

[22] The method of [20], wherein the polypeptide comprises any one of the amino acid sequence consisting of RRGPK (SEQ ID NO: 104), the amino acid sequence consisting of RRGPS (SEQ ID NO: 117), the amino acid sequence consisting of LHEALHAHYTRKE (SEQ ID NO: 105), and the amino acid sequence consisting of LHEALHAHTTRKE (SEQ ID NO: 118).

[23] The method of [22], wherein the polypeptide comprises a modified IgG heavy chain constant region that comprises any one of the amino acid sequence consisting of RRGPK (SEQ ID NO: 104), the amino acid sequence consisting of RRGPS (SEQ ID NO: 117), the amino acid sequence consisting of LHEALHAHYTRKE (SEQ ID NO: 105), and the amino acid sequence consisting of LHEALHAHTTRKE (SEQ ID NO: 118).

[24] A method for measuring concentration of a first antibody in a sample, wherein the first antibody can bind to a first epitope of an antigen, wherein the sample comprises the first antibody and the antigen, and wherein the method comprises
(A) contacting the sample with a plate or beads on which a second antibody is immobilized,
(B) contacting a solution that comprises the antigen and does not comprise the first antibody and the second antibody with the plate or the beads after (A), and
(C) detecting the antigen that is captured on the plate or the beads via the second antibody and the first antibody by using a third antibody after (B), wherein
   the first antibody comprises a modified IgG heavy chain constant region that is derived from any one of constant regions in human naturally occurring IgGs or that is derived from a chimeric constant region obtained from at least two selected from the constant regions in human naturally occurring IgGs, wherein the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system), the second antibody is the antibody of any one of [1] to [15], and the third antibody can bind to a second epitope of the antigen which is different from the first epitope and has an IgG heavy chain constant region whose amino acid sequence is different from those of the first antibody and the second antibody.

[25] A method for measuring concentration of an antigen in a sample, wherein the sample comprises the antigen and a first antibody that can bind to a first epitope of the antigen, wherein the method comprises
(D) contacting the sample with a plate or beads on which a third antibody is immobilized,
(E) contacting a solution that comprises the first antibody and does not comprise the antigen and the third antibody with the plate or the beads after (D), and
(F) detecting the first antibody that is captured on the plate or the beads via the third antibody and the antigen by using a second antibody, wherein
   the first antibody comprises a modified IgG heavy chain constant region that is derived from any one of constant regions in human naturally occurring IgGs or that is derived from a chimeric constant region obtained from at least two selected from the constant regions in human naturally occurring IgGs, wherein the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system),
   the second antibody is the antibody of any one of [1] to [15] and has an IgG heavy chain constant region whose amino acid sequence is different from those of the first antibody and the third antibody, and
   the third antibody can bind to a second epitope of the antigen which is different from the first epitope.

### BRIEF DESCRIPTION OF DRAWINGS

FIG. 1 illustrates sequence alignment of 5 modified IgG heavy chain constant regions (SG115 (SEQ ID NO:1), SG115v1 (SEQ ID NO:2), SG115v2 (SEQ ID NO:3), G1m (SEQ ID NO:4), and G4d (SEQ ID NO:5) described in Example 2. Human IgG CH germline sequences, namely IGHG1_01 (J00228) (SEQ ID NO:119) and IGHG4_01 (K01316) (SEQ ID NO:120), are also aligned for comparison. A dot indicates the same amino acid as SG115 at the position.

FIG. 2-1 and FIG. 2-2 illustrate the binding of 12 Anti-SG115 Antibodies to 5 modified IgG heavy chain constant regions (SG115, SG115v1, SG115v2, G1m, and G4d) in ELISA. SKA0009, SKA0016, SKA0046, SKA0052, SKA0054, and SKA0127 showed selective binding to SG115v1, while SKA0001, SKA0027, SKA0028, SKA0117, SKA0141, and SKA0171 showed selective binding to SG115v2, as described in Example 2.

FIG. 2-2 is a continuation of FIG. 2-1.

FIG. 3 illustrates a scheme of Fc-Mutated Antibody detection assay.

FIG. 4 illustrates a scheme of antigen detection assay.

FIG. 5 illustrates a scheme of Simoa (registered trademark) assay.

FIG. 6 illustrates the sensorgram of dissociation of human C5 from Anti-hC5 Antibody that was captured by SKA0016 and SKA0117 at pH7.4 and pH6.0. Both SKA0016 and SKA0117 did not disturb the pH-dependent interaction between Anti-hC5 Antibody and human C5.

FIG. 7 illustrates the sensorgram of binding analysis of human Fc receptor to Anti-hC5 Antibody that was captured by SKA0016. SKA0016 did not interrupt the binding between hFcRn and Anti-hC5 Antibody.

### DESCRIPTION OF EMBODIMENTS

### I. Definition

“Affinity” refers to the strength of the sum total of noncovalent interactions between a single binding site of a molecule (e.g., an antibody) and its binding partner (e.g., an antigen). Unless indicated otherwise, as used herein, “binding affinity” or “binding activity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

The term “an isolated antibody which specifically binds to a modified IgG heavy chain constant region that is derived from any one of constant regions in human naturally occurring IgGs or that is derived from a chimeric constant region obtained from at least two selected from the constant regions in human naturally occurring IgGs” refers to an antibody that is capable of binding to a specific type of modified IgG heavy chain constant region with sufficient affinity such that the antibody is useful as a detection, capturing, or diagnostic agent in targeting the modified IgG heavy chain constant region. In one embodiment, for an antibody that specifically binds to the modified IgG heavy chain constant region, the extent of binding of the antibody to a non-modified human IgG heavy chain constant region is less than about 10% of the binding of the modified IgG heavy chain constant region as measured, e.g., by a radioimmunoassay (RIA). In certain embodiments, an antibody that binds to the modified IgG heavy chain constant region has a dissociation constant (Kd) of 1 micro M or less, 100 nM or less, 10 nM or less, 1 nM or less, 0.1 nM or less, 0.01 nM or less, or 0.001 nM or less (e.g., 10-8 M or less, e.g. from 10-8 M to 10-13 M, e.g., from 10-9 M to 10-13 M). In certain embodiments, an antibody that binds to the modified IgG heavy chain constant region binds to an epitope in the modified IgG heavy chain constant region.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “constant region” herein is a region in an antibody corresponding to any one of an IgG1 constant region consisting of the amino acid sequence of SEQ ID NO: 106, an IgG2 constant region consisting of the amino acid sequence of SEQ ID NO: 107, an IgG3 constant region consisting of the amino acid sequence of SEQ ID NO: 108, and an IgG4 constant region consisting of the amino acid sequence of SEQ ID NO: 109. The constant region consists of a CH1 region (positions 118 to 215 according to the EU numbering system), a hinge region (positions 216 to 230 according to the EU numbering system), a CH2 region (positions 231 to 340 according to the EU numbering system), and a CH3 region (positions 341 to 446 according to the EU numbering system).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

### II. Antibody

An antibody in the present invention is an isolated antibody which specifically binds to a modified IgG heavy chain constant region.

In one embodiment, the antibody substantially does not bind to constant regions in human naturally occurring IgGs and a chimeric constant region obtained from at least two selected from the constant regions in human naturally occurring IgGs. In this embodiment, a binding activity of the antibody to said constant regions in human naturally occurring IgGs and the chimeric IgG consisting of at least two IgGs selected from the human naturally occurring IgGs is below the detection limit in an enzyme-linked immunoassay. On the other hand, a binding activity of the antibody to the modified IgG heavy chain constant region is detectable in an enzyme-linked immunoassay.

In a further aspect of the invention, the antibody is a monoclonal antibody, including a chimeric, humanized or human antibody. In one embodiment, the antibody is an antibody fragment, e.g., a Fv, Fab, Fab′, scFv, diabody, or F(ab′) 2 fragment. In another embodiment, the antibody is a full length antibody, e.g., an intact IgG1, IgG2, IgG3 and IgG4 antibodies, or other antibody class or isotype as defined herein.

### A. Modified IgG Heavy Chain Constant Region

In one embodiment, the modified IgG heavy chain constant region is derived from any one of constant regions in human naturally occurring IgGs or is derived from a chimeric constant region obtained from at least two selected from the constant regions in human naturally occurring IgGs. The constant regions in human naturally occurring IgGs are an IgG1 constant region consisting of the amino acid sequence of SEQ ID NO: 106, an IgG2 constant region consisting of the amino acid sequence of SEQ ID NO: 107, an IgG3 constant region consisting of the amino acid sequence of SEQ ID NO: 108, and an IgG4 constant region consisting of the amino acid sequence of SEQ ID NO: 109.

In one embodiment, the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

In further embodiment, the modified IgG heavy chain constant region can be derived from a chimeric constant region obtained from constant regions in human naturally occurring IgG1 and IgG4. In preferred embodiment, the constant region is derived from a chimeric constant region obtained from constant regions in human naturally occurring IgG1 and IgG4.

In preferred embodiment, the modified IgG heavy chain constant region comprises at least one amino acid selected from the group consisting of Arg at position 235, Arg at position 236, Lys at position 239, Leu at position 428, Ala at position 434, Arg at position 438, and Glu at position 440 (all positions according to the EU numbering system).

In one embodiment, the modified IgG heavy chain constant region may form a dimer, such as a heavy chain constant region in a naturally occurring IgG, or may form a halfmer, such as a heavy chain constant region in the monomeric Fc reported in Ishino, T. et al., J. Biol. Chem. 288:16529-37 (2013).

In one embodiment, when the modified IgG heavy chain constant region is in a human modified IgG heavy chain, the human modified IgG heavy chain is selected from the group consisting of human modified IgG1, IgG2, IgG3, and IgG4 heavy chains, and their chimeric IgG heavy chain. In a preferred embodiment, the human IgG heavy chain is a human IgG1 heavy chain, human IgG4 heavy chain, or their chimeric IgG heavy chain.

### B. Exemplary Antibodies that Specifically Recognize the Modifications Unique to a CH2 Region of the Modified IgG Heavy Chain Constant Region

Arg at position 235, Arg at position 236, and Lys at position 239 (all positions according to the EU numbering system) are those which specifically exist in CH2 region of SG115 and SG115v1 used in the Examples. Thus, the modified IgG heavy chain constant region in exemplary antibodies here preferably comprises at least a region corresponding to a CH2 region of any one of constant regions in human naturally occurring IgGs or a chimeric constant region obtained from at least two selected from the constant regions in human naturally occurring IgGs.

In one embodiment, the modified IgG heavy chain constant region comprises at least one amino acid selected from the group consisting of Arg at position 235, Arg at position 236, and Lys at position 239 (all positions according to the EU numbering system). In this embodiment, the modified IgG heavy chain constant region comprises Arg at position 235, and either or both of Arg at position 236 and Lys at position 239 (all positions according to the EU numbering system).

In preferred embodiment, the modified IgG heavy chain constant region comprises all of these three mutations, or Arg at position 235 and Arg at position 236 (both positions according to the EU numbering system). In that case, the antibody binds to the part consisting of the amino acid sequence RRGPK (SEQ ID NO: 104) or RRGPS (SEQ ID NO: 117) in the modified IgG heavy chain constant region.

In one aspect where the antibody specifically binds to the modified IgG heavy chain constant region that comprises at least one amino acid selected from the group consisting of Arg at position 235, Arg at position 236, and Lys at position 239 (all positions according to the EU numbering system), the modified IgG heavy chain constant region that comprises Arg at position 235, and either or both of Arg at position 236 and Lys at position 239 (all positions according to the EU numbering system), or the part consisting of the amino acid sequence RRGPK (SEQ ID NO: 104) or RRGPS (SEQ ID NO: 117) in the modified IgG heavy chain constant region, the invention provides an antibody comprising at least one, two, three, four, five, or six HVRs selected from (i) HVR-H1 comprising the amino acid sequence of SEQ ID NO: 33, 34, 37, 38, 39 or 41; (ii) HVR-H2 comprising the amino acid sequence of SEQ ID NO: 45, 46, 49, 50, 51 or 53; (iii) HVR-H3 comprising the amino acid sequence of SEQ ID NO: 57, 58, 61, 62, 63 or 65; (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence of SEQ ID NO: 93, 94, 97, 98, 99 or 101.

In another aspect, the invention provides an antibody comprising at least one, at least two, or all three VH HVR sequences selected from (i) HVR-H1 comprising the amino acid sequence of SEQ ID NO: 33, 34, 37, 38, 39 or 41; (ii) HVR-H2 comprising the amino acid sequence of SEQ ID NO: 45, 46, 49, 50, 51 or 53; and (iii) HVR-H3 comprising the amino acid sequence of SEQ ID NO: 57, 58, 61, 62, 63 or 65. In one embodiment, the antibody comprises HVR-H3 comprising the amino acid sequence of SEQ ID NO: 57, 58, 61, 62, 63 or 65. In another embodiment, the antibody comprises HVR-H3 comprising the amino acid sequence of SEQ ID NO: 57, 58, 61, 62, 63 or 65 and HVR-L3 comprising the amino acid sequence of SEQ ID NO: 93, 94, 97, 98, 99 or 101. In a further embodiment, the antibody comprises HVR-H3 comprising the amino acid sequence of SEQ ID NO: 57, 58, 61, 62, 63 or 65, HVR-L3 comprising the amino acid sequence of SEQ ID NO: 93, 94, 97, 98, 99 or 101, and HVR-H2 comprising the amino acid sequence of SEQ ID NO: 45, 46, 49, 50, 51 or 53. In a further embodiment, the antibody comprises (i) HVR-H1 comprising the amino acid sequence of SEQ ID NO: 33, 34, 37, 38, 39 or 41; (ii) HVR-H2 comprising the amino acid sequence of SEQ ID NO: 45, 46, 49, 50, 51 or 53; and (iii) HVR-H3 comprising the amino acid sequence of SEQ ID NO: 57, 58, 61, 62, 63 or 65.

In another aspect, the invention provides an antibody comprising at least one, at least two, or all three VL HVR sequences selected from (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence of SEQ ID NO: 93, 94, 97, 98, 99 or 101. In one embodiment, the antibody comprises (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence of SEQ ID NO: 93, 94, 97, 98, 99 or 101.

In another aspect, an antibody of the invention comprises (I) a VH domain comprising at least one, at least two, or all three VH HVR sequences selected from (i) HVR-H1 comprising the amino acid sequence of SEQ ID NO: 33, 34, 37, 38, 39 or 41, (ii) HVR-H2 comprising the amino acid sequence of SEQ ID NO: 45, 46, 49, 50, 51 or 53, and (iii) HVR-H3 comprising the amino acid sequence of SEQ ID NO: 57, 58, 61, 62, 63 or 65; and (II) a VL domain comprising at least one, at least two, or all three VL HVR sequences selected from (iv) HVR-L1 comprising the amino acid sequence of SEQ ID NO: 69, 70, 73, 74, 75 or 77, (v) HVR-L2 comprising the amino acid sequence of SEQ ID NO: 81, 82, 85, 86, 87 or 89, and (vi) HVR-L3 comprising the amino acid sequence of SEQ ID NO: 93, 94, 97, 98, 99 or 101.

In another aspect, the invention provides an antibody comprising (i) HVR-H1 comprising the amino acid sequence of SEQ ID NO: 33, 34, 37, 38, 39 or 41; (ii) HVR-H2 comprising the amino acid sequence of SEQ ID NO: 45, 46, 49, 50, 51 or 53; (iii) HVR-H3 comprising the amino acid sequence of SEQ ID NO: 57, 58, 61, 62, 63 or 65; (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence selected from SEQ ID NO: 93, 94, 97, 98, 99 or 101.

In another aspect, an antibody described herein comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 9, 10, 13, 14, 15 or 17. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody described herein comprising that sequence retains the ability to bind to a first modified IgG heavy chain constant region. In certain embodiments, a total of 1 to 10 amino acids have been substituted, inserted and/or deleted in SEQ ID NO: 9, 10, 13, 14, 15 or 17. In certain embodiments, substitutions, insertions, or deletions occur in regions outside the HVRs (i.e., in the FRs). Optionally, the antibody comprises the VH sequence in SEQ ID NO: 9, 10, 13, 14, 15 or 17, including post-translational modifications of that sequence. In a particular embodiment, the VH comprises one, two or three HVRs selected from: (i) HVR-H1 comprising the amino acid sequence of SEQ ID NO: 33, 34, 37, 38, 39 or 41, (ii) HVR-H2 comprising the amino acid sequence of SEQ ID NO: 45, 46, 49, 50, 51 or 53, and (iii) HVR-H3 comprising the amino acid sequence of SEQ ID NO: 57, 58, 61, 62, 63 or 65. Post-translational modifications include but are not limited to a modification of glutamine or glutamate in N-terminal of heavy chain or light chain to pyroglutamic acid by pyroglutamylation.

In another aspect, an antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 21, 22, 25, 26, 27 or 29. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody comprising that sequence retains the ability to bind to a first modified IgG heavy chain constant region. In certain embodiments, a total of 1 to 10 amino acids have been substituted, inserted and/or deleted in SEQ ID NO: 21, 22, 25, 26, 27 or 29. In certain embodiments, the substitutions, insertions, or deletions occur in regions outside the HVRs (i.e., in the FRs). Optionally, the antibody comprises the VL sequence in SEQ ID NO: 21, 22, 25, 26, 27 or 29, including post-translational modifications of that sequence. In a particular embodiment, the VL comprises one, two or three HVRs selected from (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence of SEQ ID NO: 93, 94, 97, 98, 99 or 101. Post-translational modifications include but are not limited to a modification of glutamine or glutamate in N-terminal of heavy chain or light chain to pyroglutamic acid by pyroglutamylation.

In another aspect, an antibody is provided, wherein the antibody comprises a VH as in any of the embodiments provided above, and a VL as in any of the embodiments provided above. In one embodiment, the antibody comprises the VH and VL sequences in SEQ ID NO: 9, 10, 13, 14, 15 or 17 and SEQ ID NO: 21, 22, 25, 26, 27 or 29, respectively, including post-translational modifications of those sequences. Post-translational modifications include but are not limited to a modification of glutamine or glutamate in N-terminal of heavy chain or light chain to pyroglutamic acid by pyroglutamylation.

In one aspect, an antibody is provided, wherein the antibody competes for binding to the first modified IgG heavy chain constant region with an antibody which comprises: (i) HVR-H1 comprising the amino acid sequence of SEQ ID NO: 33, 34, 37, 38, 39 or 41; (ii) HVR-H2 comprising the amino acid sequence of SEQ ID NO: 45, 46, 49, 50, 51 or 53; (iii) HVR-H3 comprising the amino acid sequence of SEQ ID NO: 57, 58, 61, 62, 63 or 65; (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence of SEQ ID NO: 93, 94, 97, 98, 99 or 101.

In one aspect, an antibody is provided, wherein the antibody binds to the same epitope as an antibody which comprises: (i) HVR-H1 comprising the amino acid sequence of SEQ ID NO: 33, 34, 37, 38, 39 or 41; (ii) HVR-H2 comprising the amino acid sequence of SEQ ID NO: 45, 46, 49, 50, 51 or 53; (iii) HVR-H3 comprising the amino acid sequence of SEQ ID NO: 57, 58, 61, 62, 63 or 65; (iv) HVR-L1 comprising the amino acid sequence of SEQ ID NO: 69, 70, 73, 74, 75 or 77; (v) HVR-L2 comprising the amino acid sequence of SEQ ID NO: 81, 82, 85, 86, 87 or 89; and (vi) HVR-L3 comprising the amino acid sequence of SEQ ID NO: 93, 94, 97, 98, 99 or 101.

In specific embodiments where the antibody specifically binds to the modified IgG heavy chain constant region that comprises at least one amino acid selected from the group consisting of Arg at position 235, Arg at position 236, and Lys at position 239 (all positions according to the EU numbering system), the modified IgG heavy chain constant region that comprises Arg at position 235, and either or both of Arg at position 236 and Lys at position 239 (all positions according to the EU numbering system), or the part consisting of the amino acid sequence RRGPK (SEQ ID NO: 104) or RRGPS (SEQ ID NO: 117) in the modified IgG heavy chain constant region, the antibody comprises any one of following (a) to (f):
(a) variable regions that comprise
   HVR-H1 comprising the amino acid sequence of SEQ ID NO: 33,
   HVR-H2 comprising the amino acid sequence of SEQ ID NO: 45,
   HVR-H3 comprising the amino acid sequence of SEQ ID NO: 57,
   HVR-L1 comprising the amino acid sequence of SEQ ID NO: 69,
   HVR-L2 comprising the amino acid sequence of SEQ ID NO: 81, and
   HVR-L3 comprising the amino acid sequence of SEQ ID NO: 93;
(b) variable regions that comprise
   HVR-H1 comprising the amino acid sequence of SEQ ID NO: 34,
   HVR-H2 comprising the amino acid sequence of SEQ ID NO: 46,
   HVR-H3 comprising the amino acid sequence of SEQ ID NO: 58,
   HVR-L1 comprising the amino acid sequence of SEQ ID NO: 70,
   HVR-L2 comprising the amino acid sequence of SEQ ID NO: 82, and
   HVR-L3 comprising the amino acid sequence of SEQ ID NO: 94;
(c) variable regions that comprise
   HVR-H1 comprising the amino acid sequence of SEQ ID NO: 37,
   HVR-H2 comprising the amino acid sequence of SEQ ID NO: 49,
   HVR-H3 comprising the amino acid sequence of SEQ ID NO: 61,
   HVR-L1 comprising the amino acid sequence of SEQ ID NO: 73,
   HVR-L2 comprising the amino acid sequence of SEQ ID NO: 85, and
   HVR-L3 comprising the amino acid sequence of SEQ ID NO: 97;
(d) variable regions that comprise
   HVR-H1 comprising the amino acid sequence of SEQ ID NO: 38,
   HVR-H2 comprising the amino acid sequence of SEQ ID NO: 50,
   HVR-H3 comprising the amino acid sequence of SEQ ID NO: 62,
   HVR-L1 comprising the amino acid sequence of SEQ ID NO: 74,
   HVR-L2 comprising the amino acid sequence of SEQ ID NO: 86, and
   HVR-L3 comprising the amino acid sequence of SEQ ID NO: 98;
(e) variable regions that comprise
   HVR-H1 comprising the amino acid sequence of SEQ ID NO: 39,
   HVR-H2 comprising the amino acid sequence of SEQ ID NO: 51,
   HVR-H3 comprising the amino acid sequence of SEQ ID NO: 63,
   HVR-L1 comprising the amino acid sequence of SEQ ID NO: 75,
   HVR-L2 comprising the amino acid sequence of SEQ ID NO: 87, and
   HVR-L3 comprising the amino acid sequence of SEQ ID NO: 99; and
(f) variable regions that comprise
   HVR-H1 comprising the amino acid sequence of SEQ ID NO: 41,
   HVR-H2 comprising the amino acid sequence of SEQ ID NO: 53,
   HVR-H3 comprising the amino acid sequence of SEQ ID NO: 65,
   HVR-L1 comprising the amino acid sequence of SEQ ID NO: 77,
   HVR-L2 comprising the amino acid sequence of SEQ ID NO: 89, and
   HVR-L3 comprising the amino acid sequence of SEQ ID NO: 101.

### C. Exemplary Antibodies that Specifically Recognize the Modifications Unique to a CH3 Region of the Modified IgG Heavy Chain Constant Region

Leu at position 428, Ala at position 434, Arg at position 438, and Glu at position 440 (all positions according to the EU numbering system) are those which specifically exist in CH3 region of SG115 and SG115v2 used in the Examples. Thus, the modified IgG heavy chain constant region in exemplary antibodies here preferably comprises at least a region corresponding to a CH3 region of any one of constant regions in human naturally occurring IgGs or a chimeric constant region obtained from at least two selected from the constant regions in human naturally occurring IgGs.

In one embodiment, the modified IgG heavy chain constant region comprises at least one selected from the group consisting of Leu at position 428, Ala at position 434, Arg at position 438, and Glu at position 440 (all positions according to the EU numbering system). In this embodiment, the modified IgG heavy chain constant region comprises Leu at position 428, Ala at position 434, Arg at position 438, and Glu at position 440, and optionally threonine at position 436 (all positions according to the EU numbering system).

In preferred embodiment, the modified IgG heavy chain constant region comprises all of these mutations. In that case, the antibody binds to the part consisting of the amino acid sequence LHEALHAHYTRKE (SEQ ID NO: 105) or LHEALHAHTTRKE (SEQ ID NO: 118) in the modified IgG heavy chain constant region.

In one aspect where the antibody specifically binds to the modified IgG heavy chain constant region that comprises at least one selected from the group consisting of Leu at position 428, Ala at position 434, Arg at position 438, and Glu at position 440 (all positions according to the EU numbering system), the modified IgG heavy chain constant region that comprises Leu at position 428, Ala at position 434, Arg at position 438, and Glu at position 440 (all positions according to the EU numbering system), or the part consisting of the amino acid sequence LHEALHAHYTRKE (SEQ ID NO: 105) or LHEALHAHTTRKE (SEQ ID NO: 118) in the modified IgG heavy chain constant region, the invention provides an antibody comprising at least one, two, three, four, five, or six HVRs selected from (i) HVR-H1 comprising the amino acid sequence of SEQ ID NO: 32, 35, 36, 40, 42 or 43; (ii) HVR-H2 comprising the amino acid sequence of SEQ ID NO: 44, 47, 48, 52, 54 or 55; (iii) HVR-H3 comprising the amino acid sequence of SEQ ID NO: 56, 59, 60, 64, 66 or 67; (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence of SEQ ID NO: 92, 95, 96, 100, 102 or 103.

In one aspect, the invention provides an antibody comprising at least one, at least two, or all three VH HVR sequences selected from (i) HVR-H1 comprising the amino acid sequence of SEQ ID NO: 32, 35, 36, 40, 42 or 43; (ii) HVR-H2 comprising the amino acid sequence of SEQ ID NO: 44, 47, 48, 52, 54 or 55; and (iii) HVR-H3 comprising the amino acid sequence of SEQ ID NO: 56, 59, 60, 64, 66 or 67. In one embodiment, the antibody comprises HVR-H3 comprising the amino acid sequence of SEQ ID NO: 56, 59, 60, 64, 66 or 67. In another embodiment, the antibody comprises HVR-H3 comprising the amino acid sequence of SEQ ID NO: 56, 59, 60, 64, 66 or 67 and HVR-L3 comprising the amino acid sequence of SEQ ID NO: 92, 95, 96, 100, 102 or 103. In a further embodiment, the antibody comprises HVR-H3 comprising the amino acid sequence of SEQ ID NO: 56, 59, 60, 64, 66 or 67, HVR-L3 comprising the amino acid sequence of SEQ ID NO: 92, 95, 96, 100, 102 or 103, and HVR-H2 comprising the amino acid sequence of SEQ ID NO: 44, 47, 48, 52, 54 or 55. In a further embodiment, the antibody comprises (i) HVR-H1 comprising the amino acid sequence of SEQ ID NO: 32, 35, 36, 40, 42 or 43; (ii) HVR-H2 comprising the amino acid sequence of SEQ ID NO: 44, 47, 48, 52, 54 or 55; and (iii) HVR-H3 comprising the amino acid sequence of SEQ ID NO: 56, 59, 60, 64, 66 or 67.

In another aspect, the invention provides an antibody comprising at least one, at least two, or all three VL HVR sequences selected from (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence of SEQ ID NO: 92, 95, 96, 100, 102 or 103. In one embodiment, the antibody comprises (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence of SEQ ID NO: 92, 95, 96, 100, 102 or 103.

In another aspect, an antibody of the invention comprises (I) a VH domain comprising at least one, at least two, or all three VH HVR sequences selected from (i) HVR-H1 comprising the amino acid sequence of SEQ ID NO: 32, 35, 36, 40, 42 or 43, (ii) HVR-H2 comprising the amino acid sequence of SEQ ID NO: 44, 47, 48, 52, 54 or 55, and (iii) HVR-H3 comprising the amino acid sequence of SEQ ID NO: 56, 59, 60, 64, 66 or 67; and (II) a VL domain comprising at least one, at least two, or all three VL HVR sequences selected from (iv) HVR-L1 comprising the amino acid sequence of SEQ ID NO: 68, 71, 72, 76, 78 or 79, (v) HVR-L2 comprising the amino acid sequence of SEQ ID NO: 80, 83, 84, 88, 90 or 91, and (vi) HVR-L3 comprising the amino acid sequence of SEQ ID NO: 92, 95, 96, 100, 102 or 103.

In another aspect, the invention provides an antibody comprising (i) HVR-H1 comprising the amino acid sequence of SEQ ID NO: 32, 35, 36, 40, 42 or 43; (ii) HVR-H2 comprising the amino acid sequence of SEQ ID NO: 44, 47, 48, 52, 54 or 55; (iii) HVR-H3 comprising the amino acid sequence of SEQ ID NO: 56, 59, 60, 64, 66 or 67; (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence selected from SEQ ID NO: 92, 95, 96, 100, 102 or 103.

In another aspect, an antibody described herein comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 8, 11, 12, 16, 18 or 19. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody described herein comprising that sequence retains the ability to bind to a first modified IgG heavy chain constant region. In certain embodiments, a total of 1 to 10 amino acids have been substituted, inserted and/or deleted in SEQ ID NO: 8, 11, 12, 16, 18 or 19. In certain embodiments, substitutions, insertions, or deletions occur in regions outside the HVRs (i.e., in the FRs). Optionally, the antibody comprises the VH sequence in SEQ ID NO: 8, 11, 12, 16, 18 or 19, including post-translational modifications of that sequence. In a particular embodiment, the VH comprises one, two or three HVRs selected from: (i) HVR-H1 comprising the amino acid sequence of SEQ ID NO: 32, 35, 36, 40, 42 or 43, (ii) HVR-H2 comprising the amino acid sequence of SEQ ID NO: 44, 47, 48, 52, 54 or 55, and (iii) HVR-H3 comprising the amino acid sequence of SEQ ID NO: 56, 59, 60, 64, 66 or 67. Post-translational modifications include but are not limited to a modification of glutamine or glutamate in N-terminal of heavy chain or light chain to pyroglutamic acid by pyroglutamylation.

In another aspect, an antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 20, 23, 24, 28, 30 or 31. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody comprising that sequence retains the ability to bind to a first modified IgG heavy chain constant region. In certain embodiments, a total of 1 to 10 amino acids have been substituted, inserted and/or deleted in SEQ ID NO: 20, 23, 24, 28, 30 or 31. In certain embodiments, the substitutions, insertions, or deletions occur in regions outside the HVRs (i.e., in the FRs). Optionally, the antibody comprises the VL sequence in SEQ ID NO: 20, 23, 24, 28, 30 or 31, including post-translational modifications of that sequence. In a particular embodiment, the VL comprises one, two or three HVRs selected from (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence of SEQ ID NO: 92, 95, 96, 100, 102 or 103. Post-translational modifications include but are not limited to a modification of glutamine or glutamate in N-terminal of heavy chain or light chain to pyroglutamic acid by pyroglutamylation.

In another aspect, an antibody is provided, wherein the antibody comprises a VH as in any of the embodiments provided above, and a VL as in any of the embodiments provided above. In one embodiment, the antibody comprises the VH and VL sequences in SEQ ID NO: 8, 11, 12, 16, 18 or 19 and SEQ ID NO: 20, 23, 24, 28, 30 or 31, respectively, including post-translational modifications of those sequences. Post-translational modifications include but are not limited to a modification of glutamine or glutamate in N-terminal of heavy chain or light chain to pyroglutamic acid by pyroglutamylation.

In one aspect, an antibody is provided, wherein the antibody competes for binding to the first modified IgG heavy chain constant region with an antibody which comprises: (i) HVR-H1 comprising the amino acid sequence of SEQ ID NO: 32, 35, 36, 40, 42 or 43; (ii) HVR-H2 comprising the amino acid sequence of SEQ ID NO: 44, 47, 48, 52, 54 or 55; (iii) HVR-H3 comprising the amino acid sequence of SEQ ID NO: 56, 59, 60, 64, 66 or 67; (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence of SEQ ID NO: 92, 95, 96, 100, 102 or 103.

In one aspect, an antibody is provided, wherein the antibody binds to the same epitope as an antibody which comprises: (i) HVR-H1 comprising the amino acid sequence of SEQ ID NO: 32, 35, 36, 40, 42 or 43; (ii) HVR-H2 comprising the amino acid sequence of SEQ ID NO: 44, 47, 48, 52, 54 or 55; (iii) HVR-H3 comprising the amino acid sequence of SEQ ID NO: 56, 59, 60, 64, 66 or 67; (iv) HVR-L1 comprising the amino acid sequence of SEQ ID NO: 68, 71, 72, 76, 78 or 79; (v) HVR-L2 comprising the amino acid sequence of SEQ ID NO: 80, 83, 84, 88, 90 or 91; and (vi) HVR-L3 comprising the amino acid sequence of SEQ ID NO: 92, 95, 96, 100, 102 or 103.

In specific embodiments where the antibody specifically binds to the modified IgG heavy chain constant region that comprises at least one selected from the group consisting of Leu at position 428, Ala at position 434, Arg at position 438, and Glu at position 440 (all positions according to the EU numbering system), the modified IgG heavy chain constant region that comprises Leu at position 428, Ala at position 434, Arg at position 438, and Glu at position 440 (all positions according to the EU numbering system), or the part consisting of the amino acid sequence LHEALHAHYTRKE (SEQ ID NO: 105) or LHEALHAHTTRKE (SEQ ID NO: 118) in the modified IgG heavy chain constant region, the antibody comprises any one of following (g) to (l):
(g) variable regions that comprise
   HVR-H1 comprising the amino acid sequence of SEQ ID NO: 32,
   HVR-H2 comprising the amino acid sequence of SEQ ID NO: 44,
   HVR-H3 comprising the amino acid sequence of SEQ ID NO: 56,
   HVR-L1 comprising the amino acid sequence of SEQ ID NO: 68,
   HVR-L2 comprising the amino acid sequence of SEQ ID NO: 80, and
   HVR-L3 comprising the amino acid sequence of SEQ ID NO: 92;
(h) variable regions that comprise
   HVR-H1 comprising the amino acid sequence of SEQ ID NO: 35,
   HVR-H2 comprising the amino acid sequence of SEQ ID NO: 47,
   HVR-H3 comprising the amino acid sequence of SEQ ID NO: 59,
   HVR-L1 comprising the amino acid sequence of SEQ ID NO: 71,
   HVR-L2 comprising the amino acid sequence of SEQ ID NO: 83, and
   HVR-L3 comprising the amino acid sequence of SEQ ID NO: 95;
(i) variable regions that comprise
   HVR-H1 comprising the amino acid sequence of SEQ ID NO: 36,
   HVR-H2 comprising the amino acid sequence of SEQ ID NO: 48,
   HVR-H3 comprising the amino acid sequence of SEQ ID NO: 60,
   HVR-L1 comprising the amino acid sequence of SEQ ID NO: 72,
   HVR-L2 comprising the amino acid sequence of SEQ ID NO: 84, and
   HVR-L3 comprising the amino acid sequence of SEQ ID NO: 96;
(j) variable regions that comprise
   HVR-H1 comprising the amino acid sequence of SEQ ID NO: 40,
   HVR-H2 comprising the amino acid sequence of SEQ ID NO: 52,
   HVR-H3 comprising the amino acid sequence of SEQ ID NO: 64,
   HVR-L1 comprising the amino acid sequence of SEQ ID NO: 76,
   HVR-L2 comprising the amino acid sequence of SEQ ID NO: 88, and
   HVR-L3 comprising the amino acid sequence of SEQ ID NO: 100;
(k) variable regions that comprise
   HVR-H1 comprising the amino acid sequence of SEQ ID NO: 42,
   HVR-H2 comprising the amino acid sequence of SEQ ID NO: 54,
   HVR-H3 comprising the amino acid sequence of SEQ ID NO: 66,
   HVR-L1 comprising the amino acid sequence of SEQ ID NO: 78,
   HVR-L2 comprising the amino acid sequence of SEQ ID NO: 90, and
   HVR-L3 comprising the amino acid sequence of SEQ ID NO: 102; and
(l) variable regions that comprise
   HVR-H1 comprising the amino acid sequence of SEQ ID NO: 43,
   HVR-H2 comprising the amino acid sequence of SEQ ID NO: 55,
   HVR-H3 comprising the amino acid sequence of SEQ ID NO: 67,
   HVR-L1 comprising the amino acid sequence of SEQ ID NO: 79,
   HVR-L2 comprising the amino acid sequence of SEQ ID NO: 91, and
   HVR-L3 comprising the amino acid sequence of SEQ ID NO: 103.

### D. Other Embodiments

In one embodiment, the antibody in the present invention encompasses an antibody which binds to the same epitope as any one of the antibodies mentioned in the above sections, “A. Modified IgG heavy chain constant region” to “C. Exemplary antibodies that specifically recognize the modifications unique to a CH3 region of the modified IgG heavy chain constant region”.

In one embodiment, the antibody in the present invention encompasses an antibody which specifically binds to a modified IgG heavy chain constant region, wherein binding of the antibody to the modified IgG heavy chain constant region competes with the antibodies mentioned in the above sections, “A. Modified IgG heavy chain constant region” to “C. Exemplary antibodies that specifically recognize the modifications unique to a CH3 region of the modified IgG heavy chain constant region”. In the embodiment, the modified IgG heavy chain constant region is derived from any one of constant regions in human naturally occurring IgGs or is derived from a chimeric constant region obtained from at least two selected from the constant regions in human naturally occurring IgGs. The modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system). In the embodiment, specific antibodies referred to herein are the same as those in the above sections.

### E. Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an antibody described herein is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody described herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### F. Assays

Antibodies provided herein may be identified, screened for, or characterized for their physical/chemical properties and/or biological activities by various assays known in the art.

### G. Binding Assays and Other Assays

In one aspect, an antibody of the invention is tested for its antigen binding activity, e.g., by known methods such as ELISA, Western blot, etc.

In another aspect, competition assays may be used to identify an antibody that competes with any one of antibodies used in the Examples (SKA0001, SKA0009, SKA0016, SKA0027, SKA0028, SKA0046, SKA0052, SKA0054, KA0117, SKA0127, SKA0141, and SKA0171) for binding to a modified IgG heavy chain constant region that is derived from any one of constant regions in human naturally occurring IgGs or that is derived from a chimeric constant region obtained from at least two selected from the constant regions in human naturally occurring IgGs, wherein the modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system). In certain embodiments, such a competing antibody may bind to the same epitope (e.g., a linear or a conformational epitope) as the epitope(s) to which any one of antibodies used in the Examples (SKA0001, SKA0009, SKA0016, SKA0027, SKA0028, SKA0046, SKA0052, SKA0054, KA0117, SKA0127, SKA0141, and SKA0171) binds. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, an immobilized modified IgG heavy chain constant region is incubated in a solution comprising a labeled antibody that binds to the modified IgG heavy chain constant region and an unlabeled antibody that is being tested for its ability to compete with the labeled antibody for binding to the immobilized modified IgG heavy chain constant region. The unlabeled antibody can be present in a B-cell or hybridoma supernatant. As a control, the immobilized modified IgG heavy chain constant region is incubated in a solution comprising the labeled antibody but not comprising the unlabeled antibody. After incubation under conditions permissive for binding of the labeled antibody to the immobilized modified IgG heavy chain constant region, excess unbound antibody is removed, and the amount of label associated with the immobilized modified IgG heavy chain constant region is measured. If the amount of label associated with the immobilized modified IgG heavy chain constant region is substantially reduced in the test sample relative to the control sample, then that indicates that the unlabeled antibody is competing with the labeled antibody for binding to the immobilized modified IgG heavy chain constant region. See Harlow and Lane (1988) Antibodies: A Laboratory Manual ch.14 (Cold Spring Harbor Laboratory, Cold Spring Harbor, NY).

### III. Composition

In one aspect, a composition in the present invention is a composition for use in detecting or capturing a polypeptide in a sample. The composition comprises any one of the antibodies described in “II. Antibody”.

In another aspect, a composition in the present invention is a composition for use in treating or preventing a disease. When the antibody is used for treatment or prevention of any disease, the composition can be or include cells that express any one of the antibodies described in “II. Antibody” or fragments thereof, which specifically bind to a modified IgG heavy chain constant region.

In a preferred embodiment, the polypeptide in the sample comprises a modified IgG heavy chain constant region that is derived from any one of constant regions in human naturally occurring IgGs or that is derived from a chimeric constant region of at least two selected from the constant regions in human naturally occurring IgGs. The modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system). Specific antibodies referred to herein are the same as those described in “II. Antibody”.

In another preferred embodiment, the polypeptide comprises any one of the amino acid sequence consisting of RRGPK (SEQ ID NO: 104), the amino acid sequence consisting RRGPS (SEQ ID NO: 117), the amino acid sequence consisting LHEALHAHYTRKE (SEQ ID NO: 105), and the amino acid sequence consisting LHEALHAHTTRKE (SEQ ID NO: 118). The polypeptide detected or captured by the composition is not particularly limited in terms of its structure, as long as the polypeptide comprises any one or more of these amino acid sequences. The polypeptide preferably comprises a modified IgG heavy chain constant region that comprises any one or more of the amino acid sequences.

In one embodiment, the polypeptide detected or captured by the composition may be an antibody such as a human IgG1, IgG2, IgG3, or IgG4 molecule, an antibody fragment, a fusion protein, or a polypeptide of any other form comprising the modified IgG heavy chain constant region or an epitope in it.

In the case that the polypeptide comprises the modified IgG heavy chain constant region, the modified IgG heavy chain constant region may comprise other amino acid substitutions or modifications as long as it comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

### IV. Method

In one aspect, a method in the present invention is a method for detecting or capturing a polypeptide in a sample. The method comprises contacting a sample with any one of the antibodies described in “II. Antibody” or with any one of the compositions described in “III. Composition”.

In a preferred embodiment, the polypeptide comprises a modified IgG heavy chain constant region. The modified IgG heavy chain constant region is derived from any one of constant regions in human naturally occurring IgGs or is derived from a chimeric constant region of at least two selected from the constant regions in human naturally occurring IgGs. The modified IgG heavy chain constant region comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

In another preferred embodiment, the polypeptide comprises any one of the amino acid sequence consisting of RRGPK (SEQ ID NO: 104), the amino acid sequence consisting of RRGPS (SEQ ID NO: 117), the amino acid sequence consisting of LHEALHAHYTRKE (SEQ ID NO: 105), and the amino acid sequence consisting of LHEALHAHTTRKE (SEQ ID NO: 118). The polypeptide detected or captured by the method is not particularly limited in terms of its structure, as long as the polypeptide comprises one or more of these amino acid sequences. The polypeptide preferably comprises a modified IgG heavy chain constant region that comprises one or more of these amino acid sequences.

In one embodiment, the polypeptide detected or captured by the method may be an antibody such as a human IgG1, IgG2, IgG3, or IgG4 molecule, an antibody fragment, a fusion protein, or a polypeptide of any other form comprising the modified IgG heavy chain constant region or an epitope in it.

In the case that the polypeptide comprises the modified IgG heavy chain constant region, the modified IgG heavy chain constant region may comprise other amino acid substitutions or modifications as long as it comprises at least one amino acid selected from the group consisting of Arg at position 235, Arg at position 236, Lys at position 239, Gly at position 327, Ser at position 330, Ser at position 331, Leu at position 428, Ala at position 434, Arg at position 438, and Glu at position 440 (all positions according to the EU numbering system).

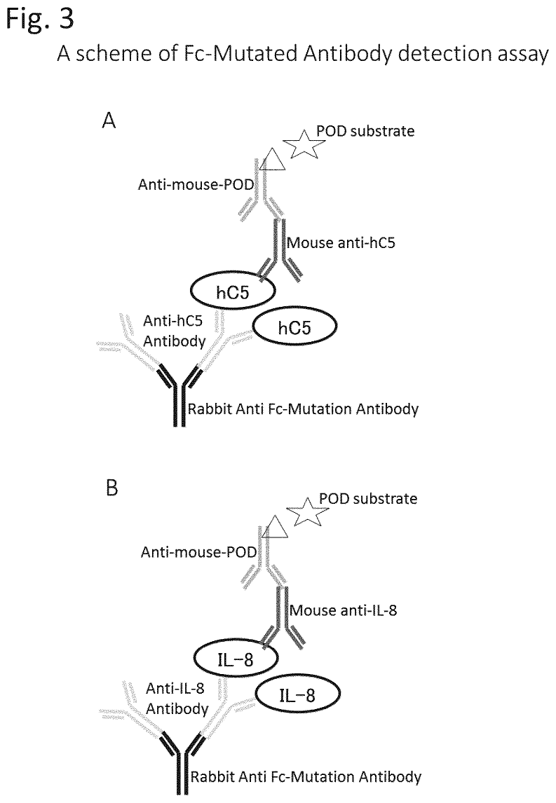

A method by ELISA is illustrated in FIG. 3 as a specific embodiment of the method. “Rabbit Anti Fc-Mutation Antibody” corresponds to one of the antibodies described in “II. Antibody”, and “Anti-hC5 Antibody” in FIG. 3A and “Anti-IL-8 Antibody” in FIG. 3B correspond to the polypeptide comprising the modified IgG heavy chain constant region. Rabbit Anti Fc-Mutation Antibody immobilized on a plate captures Anti-hC5 Antibody in FIG. 3A and Anti-IL-8 Antibody in FIG. 3B in a sample. And then hC5 (human complement 5) as an antigen of Anti-hC5 Antibody, “Mouse anti-hC5” that binds to a different epitope from the epitope to which Anti-hC5 Antibody binds, and Anti-mouse-POD are reacted in this order in FIG. 3A. In FIG. 3B, IL-8 as an antigen of Anti-IL-8 Antibody, “Mouse anti-IL-8” that binds to a different epitope from the epitope to which Anti-IL-8 Antibody binds, and Anti-mouse-POD are reacted in this order. Finally, POD substrate is added on the plate and its luminescent is measured. In this embodiment, when there is a certain amount of the polypeptide comprising the modified IgG heavy chain constant region in a sample, luminescence is detected by a luminometer.

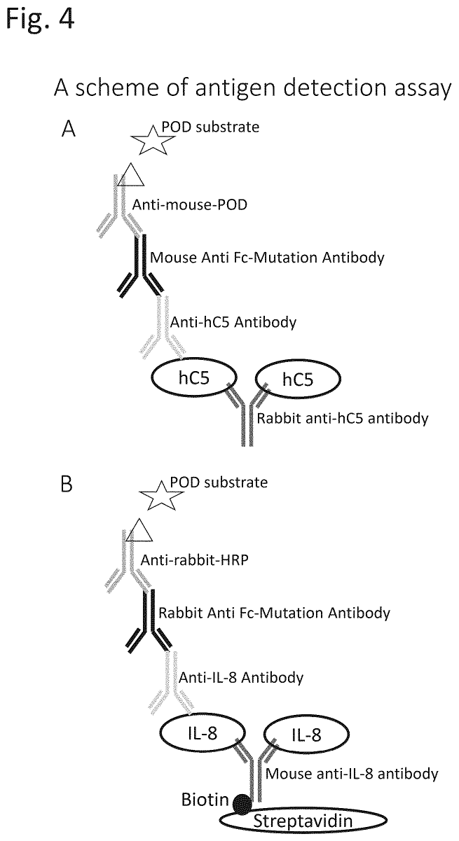

In another embodiment, the antibodies described in “II. Antibody” can be applied for detecting an antigen, such as hC5 and IL-8 as shown in FIG. 4. In this embodiment, “Mouse Anti Fc-Mutation Antibody” and “Rabbit Anti Fc-Mutation Antibody” correspond to one of the antibodies described in “II. Antibody”, and “Anti-hC5 Antibody” in FIG. 4A and “Anti-IL-8 Antibody” in FIG. 4B correspond to the polypeptide comprising the modified IgG heavy chain constant region. Rabbit anti-hC5 antibody immobilized on a plate captures hC5 in a sample in FIG. 4A. Mouse anti-IL-8 antibody immobilized on a plate captures IL-8 in a sample in FIG. 4B. And then Anti-hC5 Antibody, Mouse Anti Fc-Mutation Antibody, and Anti-mouse-POD are reacted in this order in FIG. 4A. In FIG. 4B, Anti-IL-8 Antibody, Rabbit Anti Fc-Mutation Antibody, and Anti-rabbit-HRP are reacted in this order. Finally POD (peroxidase), e.g. HRP (Horseradish peroxidase), substrate is added on the plate and its luminescent is measured. In this embodiment, when there is a certain amount of hC5 or IL-8 in a sample, luminescence is detected by a luminometer.

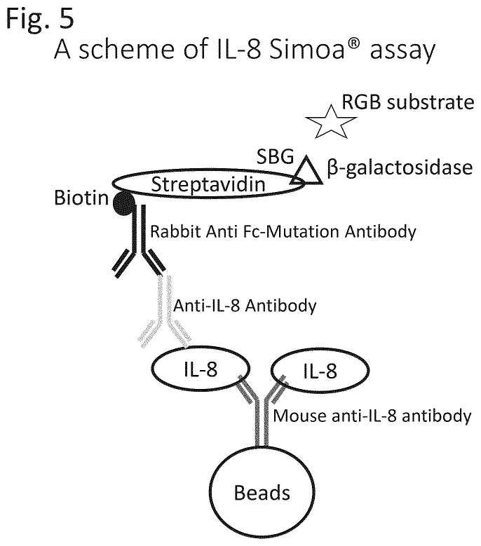

The above embodiments related to ELISA methods can be replaced by Simoa (registered trademark) assay. In one embodiment of the assay, the antibodies described in “II. Antibody” can be applied for detecting an antigen, such as IL-8 as shown in FIG. 5. In this embodiment, “Rabbit Anti Fc-Mutation Antibody” corresponds to one of the antibodies described in “II. Antibody”, and “Anti-IL-8 Antibody” corresponds to the polypeptide comprising the modified IgG heavy chain constant region. Mouse anti-IL-8 antibody immobilized on beads captures IL-8 in sample. And then Anti-IL-8 Antibody, Biotinylated Anti Fc-Mutation Antibody, and streptavidin-beta-galactosidase (SBG (Quanterix Corporation)) are reacted in this order. Finally, substrate of beta-galactocidase (RGB) is added in the reactant and its luminescence is measured.

### Example 1

Preparation of an Antibody Comprising Constant Regions that Comprise Multiple Mutations in an Fc Region

### Antibody Expression and Purification

The antibody comprising constant regions that comprise multiple mutations in an Fc region was expressed by FreeStyle293 expression system. The used constant region (SEQ ID NO: 1) is referred to as SG115 in WO2016098356A1. The harvested cell culture fluid (HCCF) was purified with rProtein A resin (MabSelect SuRe, GE) and Size exclusion chromatography (SEC, Superdex200pg, GE). In SEC process, we exchanged the buffer to 20 mmol/L Histidine, 150 mmol/L Arginine-Aspartic acid, pH6.0. Finally, the antibody was concentrated to 143 mg/mL using UF (ultrafiltration).

### Papain Digestion

For papain digestion, we used Pierce Fab Preparation Kit (Pierce, Cat. No. 44985). The papain digestion process was as described below.
The antibody concentration was adjusted to 8.0 mg/mL with digestion buffer.
   0.5 mL of the antibody solution was added to a spin column tube containing the equilibrated papain resin. The top cap and the bottom plug were placed on the spin column.
   The digestion reaction solution was incubated on a rotator for 15 hours at 37 degrees C.
   After the incubation, the bottom cap was removed and the spin column was placed into a microcentrifuge tube. The column was centrifuged at 5000×g for one minute.
   The resin was washed with 0.5 mL of Dulbecco's PBS (−). The spin column was placed into a microcentrifuge tube. The column was centrifuged at 5000×g for one minute.
   The solution of step 4 and 5 was mixed as a digested fraction. The total volume was 1.0 mL from one column.

### Purification of Fc Fragment

Papain-digested sample was purified with rProtein A resin (MabSelect SuRe, GE) and size exclusion chromatography (SEC, Superdex200 pg, GE). In the SEC process, we removed whole IgG (non-digested molecule) and exchanged the buffer to Dulbecco's PBS (−).

### Example 2

Generation of Antibodies that Recognize the Mutations in SG115

Antibodies that recognize the mutations in SG115, referred to as “Anti-SG115 Antibodies”, were prepared, selected, and assayed as described below.

Ten week old NZW rabbits were immunized intradermally with Fc fragment of SG115 (50-100 microgram/dose/rabbit). The dose was repeated 5 times over a 2-month period, and then the blood was collected from the immunized rabbits. Antigen-specific B-cells were sorted with cell sorter and then plated and cultured according to the procedure described in WO2016098356A1. After cultivation, B-cell culture supernatants were collected for further analysis and pellets were cryopreserved.

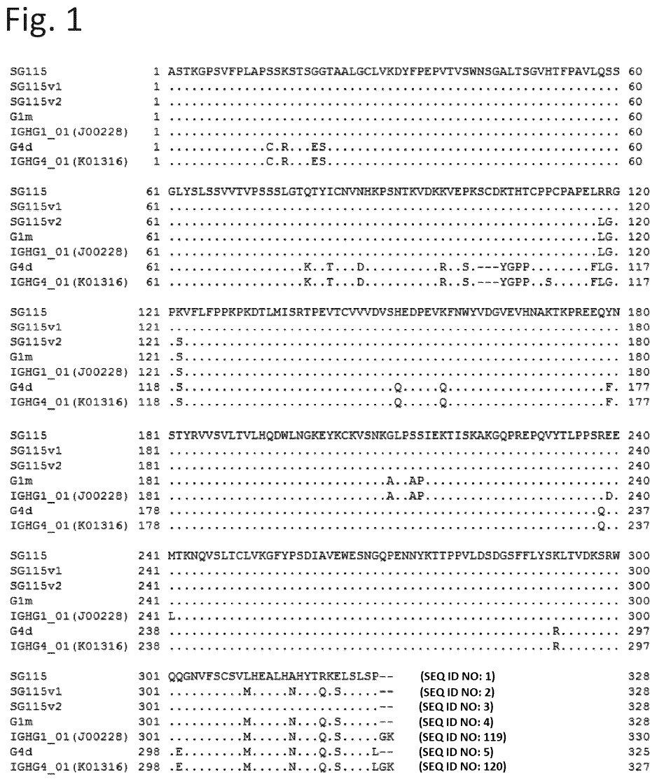

Ability to bind to SG115 was evaluated by ELISA using the B cell culture supernatants. We tested the binding to 5 kinds of modified IgG heavy chain constant regions in order to evaluate the binding specificity: SG115 (SEQ ID NO: 1), SG115v1 (SEQ ID NO: 2), SG115v2 (SEQ ID NO: 3), G1m (SEQ ID NO: 4), and G4d (SEQ ID NO: 5). The sequence alignment of these 5 constant regions is shown in FIG. 1.

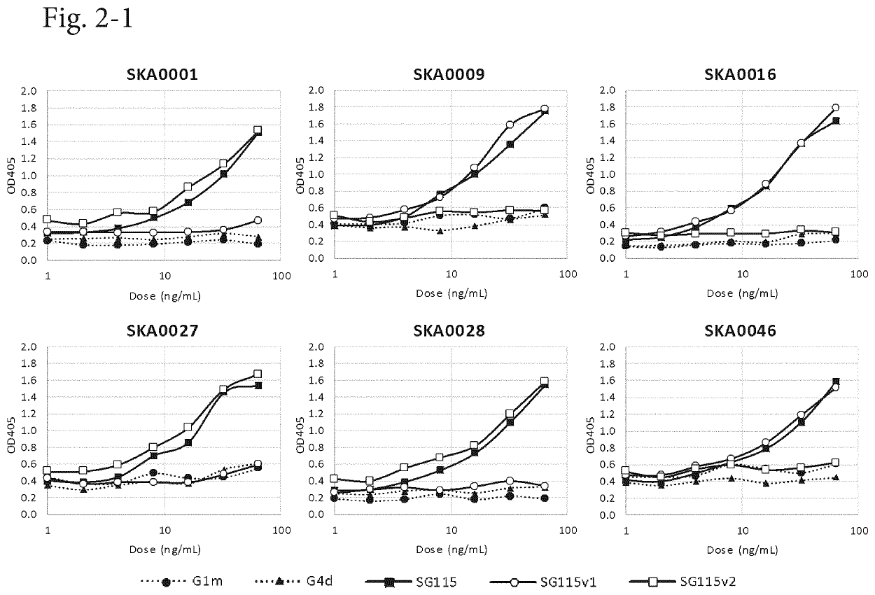

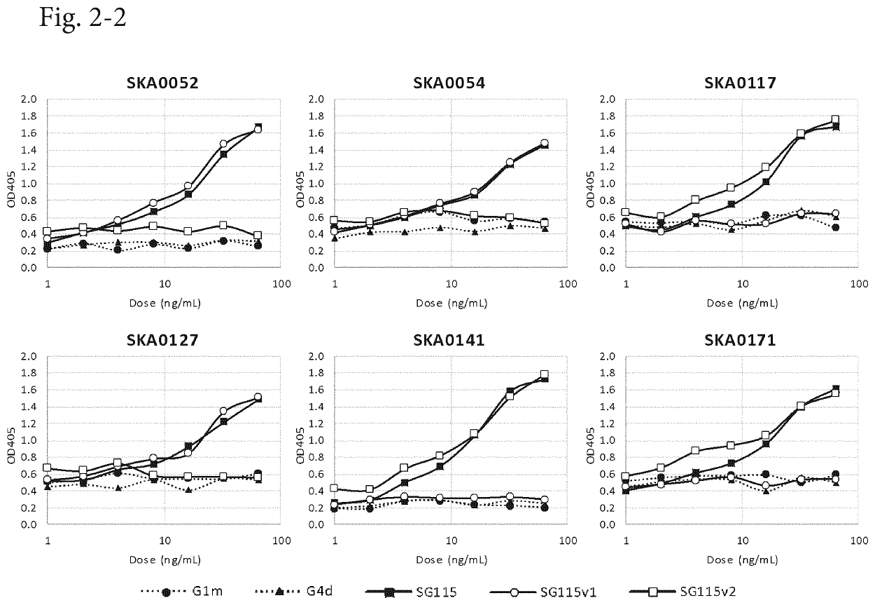

A total of 10,560 B-cell lines were screened for the binding to 5 types of modified IgG heavy chain constant regions, and 186 lines were selected and designated as SKA0001-SKA0186, which bound to SG115 but did not to G1m and G4d, and also bound to SG115v1 and/or SG115v2. RNA of the selected lines was purified from cryopreserved cell pellets using ZR-96 Quick-RNA kits (ZYMO RESEARCH, Cat No. R1053). DNA encoding antibody heavy chain variable regions in the selected lines was amplified by reverse transcription PCR and recombined with DNA encoding rbIgGv2 heavy chain constant region (SEQ ID NO: 6). DNA encoding antibody light chain variable regions was amplified by reverse transcription PCR and recombined with DNA encoding rbIgk light chain constant region (SEQ ID NO: 7). The antibodies were expressed in FreeStyle™ 293-F Cells (Invitrogen) and purified from culture supernatant. Through further evaluation, 12 clones were selected based on the binding ability and specificity in ELISA, and sequence variety of heavy chain CDR3. Of these clones, 6 clones (SKA0009, SKA0016, SKA0046, SKA0052, SKA0054 and SKA0127) showed selective binding to SG115v1 but not to SG115v2, while the other 6 clones (SKA0001, SKA0027, SKA0028, SKA0117, SKA0141 and SKA0171) showed selective binding to SG115v2 but not to SG115v1 (FIGS. 2-1 and 2-2). The VH and VL sequences of these 12 antibodies are listed in Table 1.

TABLE 1

| Antibody | Binding | SEQ ID NO. | | | | | | | |
|---|---|---|---|---|---|---|---|---|---|
| Name | Selectivity | VH | HCDR1 | HCDR2 | HCDR3 | VL | LCDR1 | LCDR2 | LCDR3 |
| SKA0001 | SG115v2 | 8 | 32 | 44 | 56 | 20 | 68 | 80 | 92 |
| SKA0009 | SG115v1 | 9 | 33 | 45 | 57 | 21 | 69 | 81 | 93 |
| SKA0016 | SG115v1 | 10 | 34 | 46 | 58 | 22 | 70 | 82 | 94 |
| SKA0027 | SG115v2 | 11 | 35 | 47 | 59 | 23 | 71 | 83 | 95 |
| SKA0028 | SG115v2 | 12 | 36 | 48 | 60 | 24 | 72 | 84 | 96 |
| SKA0046 | SG115v1 | 13 | 37 | 49 | 61 | 25 | 73 | 85 | 97 |
| SKA0052 | SG115v1 | 14 | 38 | 50 | 62 | 26 | 74 | 86 | 98 |
| SKA0054 | SG115v1 | 15 | 39 | 51 | 63 | 27 | 75 | 87 | 99 |
| SKA0117 | SG115v2 | 16 | 40 | 52 | 64 | 28 | 76 | 88 | 100 |
| SKA0127 | SG115v1 | 17 | 41 | 53 | 65 | 29 | 77 | 89 | 101 |
| SKA0141 | SG115v2 | 18 | 42 | 54 | 66 | 30 | 78 | 90 | 102 |
| SKA0171 | SG115v2 | 19 | 43 | 55 | 67 | 31 | 79 | 91 | 103 |

### Example 3

### Detection of SG115 in Sample by Anti-SG115 Antibodies

For detection of an antibody comprising an Fc region comprising all or part of the mutations in the Fc region of SG115 (hereinafter also referred to as “Fc-Mutated Antibody” or “Fc-Mutated Antibodies”) in biological sample, effectiveness of the above 12 monoclonal antibodies (hereinafter also referred to as “Anti Fc-Mutation Antibody” or “Anti Fc-Mutation Antibodies”) was evaluated. A specific anti-human C5 antibody comprising SG115 was used as a model of Fc-Mutated Antibody in EXAMPLEs 3-5, which is hereinafter referred to as “Anti-hC5 Antibody”.

### Assay Procedures

Each well of ninety six-well immunoplate was coated with a rabbit Anti Fc-Mutation Antibody and blocked with blocking buffer. Diluted serum samples were added to each well of the plate. Recombinant human C5 was added to each well of the plate. Mouse Anti-hC5 Antibody was added followed by the addition of anti-mouse-POD (Jackson ImmunoResearch Inc.). Finally, POD substrate was added to each well of the plate and OD was measured. The plate was washed between the steps.

### Reactivity Test for Antibody Selection

Twelve of the Anti Fc-Mutation Antibodies were tested. Anti-hC5 Antibody comprising SG115 was diluted with pooled human serum and measured using 12 candidates of rabbit Anti Fc-Mutation Antibodies. Signal to noise ratio was calculated. Measured OD is tabulated in Table 2. Three candidates from each epitope type (total 6 candidates) were selected for selectivity test (Table 2). That is, SKA0009, SKA0052, and SKA0127 were selected as antibodies specifically binding to SG115v1, and SKA0117, SKA0141, and SKA0171 were selected as antibodies specifically binding to SG115v2.

TABLE 2

| | | Incu- | | OD | Signal | |
|---|---|---|---|---|---|---|
| | | bation | | (Anti-hC5 | to | |
| Epitope | Antibody | period | OD | Antibody | noise | Selected |
| type | name | (min) | (Blank) | spiked) | ratio | antibodies |
| SG115v2 | SKA0001 | 18 | 0.032 | 0.079 | 2.5 | — |
| SG115v1 | SKA0009 | 9 | 0.028 | 0.089 | 3.2 | Selected |
| SG115v1 | SKA0016 | 16 | 0.030 | 0.076 | 2.5 | — |
| SG115v2 | SKA0027 | 9 | 0.027 | 0.093 | 3.4 | — |
| SG115v2 | SKA0028 | 16 | 0.030 | 0.091 | 3.0 | — |
| SG115v1 | SKA0046 | 21 | 0.032 | 0.075 | 2.3 | — |
| SG115v1 | SKA0052 | 11 | 0.027 | 0.098 | 3.7 | Selected |
| SG115v1 | SKA0054 | 19 | 0.030 | 0.077 | 2.6 | — |
| SG115v2 | SKA0117 | 9 | 0.027 | 0.112 | 4.1 | Selected |
| SG115v1 | SKA0127 | 14 | 0.028 | 0.078 | 2.8 | Selected |
| SG115v2 | SKA0141 | 8 | 0.026 | 0.109 | 4.2 | Selected |
| SG115v2 | SKA0171 | 8 | 0.027 | 0.106 | 4.0 | Selected |

### Selectivity Test for Antibody Selection

Ten individual sera with or without spiked Anti-hC5 Antibody and calibration curve samples were measured using the 6 candidate antibodies as capture reagents. Without spiked Anti-hC5 Antibody, measured concentrations of all individual samples were below limit of quantitation (BLQ) in any given rabbit Anti Fc-Mutation Antibodies. With spiked Anti-hC5 Antibody, relative error (RE) of measured concentrations of all individual samples were within +/−20% in any given rabbit Anti Fc-Mutation Antibodies (Table 3).

TABLE 3

| | | Nominal Anti-hC5 Antibody | |
|---|---|---|---|
| | | concentration: 100 ng/mL | |
| | Nominal Anti-hC5 Antibody | Measured | |
| | concentration: 0 ng/mL | concentration | |
| Antibody | Measured concentration | range in | RE range |
| name | range in serum (ng/mL) | serum (ng/mL) | (%) |
| SKA0009 | All individual | 88.5 to 95.9 | −11.5 to −4.1 |
| | samples were BLQ | | |
| SKA0052 | All individual | 93.3 to 99.2 | −6.7 to −0.8 |
| | samples were BLQ | | |
| SKA0127 | All individual | 90.4 to 99.2 | −9.6 to −0.8 |
| | samples were BLQ | | |
| SKA0141 | All individual | 91.5 to 97.0 | −8.5 to −3.0 |
| | samples were BLQ | | |
| SKA0171 | All individual | 90.5 to 97.2 | −9.5 to −2.8 |
| | samples were BLQ | | |
| SKA0117 | All individual | 87.5 to 97.8 | −12.5 to −2.2 |
| | samples were BLQ | | |

BLQ: Below the limit of quantitation

BLQ: Below the limit of quantitation

### Selected Antibody

According to the results from reactivity test and selectivity test, SKA0141 which had the highest signal to noise ratio was selected.

### Example 4

### Evaluation of the Method to Measure Anti-hC5 Antibody in Human Serum (Fc-Mutated Antibody Detection Assay)

### Assay Procedures

Ninety six well immunoplate was coated with the rabbit Anti Fc-Mutation Antibody (SKA0141) and blocked with blocking buffer. Diluted serum samples including Anti-hC5 Antibody were added to each well of the plate. Recombinant human C5 was added to each well of the plate. Mouse anti-hC5 monoclonal antibody was added followed by the addition of anti-mouse-POD (Jackson ImmunoResearch Inc.). Finally, POD substrate was added to each well of the plate and OD was measured. The plate was washed between the steps.

### Method Evaluation

Reproducibility was tested. Intra-batch accuracy (RE) and precision (CV) were −16.3% to −5.1% and 1.6% to 4.4% respectively (Table 4). Inter-batch accuracy (RE) and precision (CV) were −10.1% to −4.0% and 2.7% to 6.9% respectively (Table 5).

TABLE 4

| | Measured concentration in serum (ng/mL) | | | | |
|---|---|---|---|---|---|
| | 50 ng/mL | 100 ng/mL | 400 ng/mL | 2560 ng/mL | 3200 ng/mL |
| | 47.7 | 97.2 | 385 | 2179 | 2642 |
| | 46.4 | 94.3 | 383 | 2337 | 2698 |
| | 46.6 | 93.3 | 372 | 2176 | 2702 |
| | 48.2 | 97.7 | 383 | 2177 | 2676 |
| | 47.1 | 93.1 | 380 | 2359 | 2759 |
| | 46.2 | 93.3 | 377 | 2084 | 2504 |
| | 47.1 | 97.0 | 387 | 2137 | 2606 |
| | 46.2 | 93.6 | 364 | 2261 | 2850 |
| Mean | 46.9 | 94.9 | 379 | 2214 | 2680 |
| CV (%) | 1.6 | 2.1 | 2.0 | 4.4 | 3.8 |
| RE (%) | −6.2 | −5.1 | −5.3 | −13.5 | −16.3 |
| n | 8 | 8 | 8 | 8 | 8 |

TABLE 5
Measured concentration in serum (ng/mL)

| Assay run | 50 ng/mL | 100 ng/mL | 400 ng/mL | 2560 ng/mL | 3200 ng/mL |
|---|---|---|---|---|---|
| 1 | 47.7 | 97.2 | 385 | 2179 | 2642 |
| | 46.4 | 94.3 | 383 | 2337 | 2698 |
| | 46.6 | 93.3 | 372 | 2176 | 2702 |
| | 48.2 | 97.7 | 383 | 2177 | 2676 |
| | 47.1 | 93.1 | 380 | 2359 | 2759 |
| | 46.2 | 93.3 | 377 | 2084 | 2504 |
| | 47.1 | 97.0 | 387 | 2137 | 2606 |
| | 46.2 | 93.6 | 364 | 2261 | 2850 |
| 2 | 46.9 | 96.8 | 392 | 2215 | 2727 |
| | 46.6 | 98.7 | 401 | 2392 | 2967 |
| | 45.7 | 101 | 412 | 2315 | 3201 |
| | 47.7 | 104 | 411 | 2428 | 2992 |
| | 50.3 | 98.4 | 411 | 2650 | 3180 |
| | 50.3 | 104 | 408 | 2361 | 2975 |
| 3 | 48.3 | 93.0 | 374 | 2372 | 2938 |
| | 47.2 | 93.3 | 359 | 2412 | 3034 |
| | 48.0 | 92.7 | 376 | 2393 | 3060 |
| | 48.3 | 92.4 | 371 | 2371 | 3065 |
| | 45.4 | 91.8 | 376 | 2558 | 2992 |
| | 47.5 | 93.0 | 359 | 2345 | 2955 |
| Mean | 47.4 | 95.9 | 384 | 2326 | 2876 |
| CV (%) | 2.7 | 3.9 | 4.4 | 6.0 | 6.9 |
| RE (%) | −5.3 | −4.1 | −4.0 | −9.1 | −10.1 |
| n | 20 | 20 | 20 | 20 | 20 |

Selectivity was tested. Without spiked Anti-hC5 Antibody, measured concentrations of all individual samples were BLQ. With spiked Anti-hC5 Antibody, RE of measured concentrations of all individual samples were −15.2% to 2.2% (Table 6).

TABLE 6

| Individual | Measured concentration in serum (ng/mL) | | | | |
|---|---|---|---|---|---|
| No. | 0 ng/mL | 50.0 ng/mL | RE (%) | 3200 ng/mL | RE (%) |
| 1 | BLQ | 44.1 | −11.8 | 2855 | −10.8 |
| 2 | BLQ | 43.1 | −13.7 | 2935 | −8.3 |
| 3 | BLQ | 42.4 | −15.2 | 3024 | −5.5 |
| 4 | BLQ | 43.1 | −13.7 | 2995 | −6.4 |
| 5 | BLQ | 42.6 | −14.7 | 2927 | −8.5 |
| 6 | BLQ | 43.9 | −12.3 | 3010 | −5.9 |
| 7 | BLQ | 43.9 | −12.3 | 2987 | −6.6 |
| 8 | BLQ | 46.3 | −7.4 | 2981 | −6.8 |
| 9 | BLQ | 44.8 | −10.3 | 3091 | −3.4 |
| 10 | BLQ | 43.6 | −12.8 | 3270 | 2.2 |

Dilution linearity was tested. One milligram per milliliter of Anti-hC5 Antibody could be measured in dilution factor 50,000-fold and prozone effect was not observed. (Table 7).

TABLE 7

| | | | | Mean | | |
|---|---|---|---|---|---|---|
| Nominal | | Con- | Measured | measured | | |
| con- | | cen- | con- | con- | | |
| cen- | | tration | centration | centration | | |
| tration | Dilu- | in assay | in assay | in assay | | |
| in serum | tion | sample | sample | sample | RE | CV |
| (ng/mL) | factor | (ng/mL) | (ng/mL) | (ng/mL) | (%) | (%) |
| 1,000,000 | 50 | 20,000 | ALQ | — | — | — |
| | | | ALQ | | | |
| | | | ALQ | | | |
| | 500 | 2,000 | ALQ | — | — | — |
| | | | ALQ | | | |
| | | | ALQ | | | |
| | 5000 | 200 | ALQ | — | — | — |
| | | | ALQ | | | |
| | | | ALQ | | | |
| | 50,000 | 20 | 19.0 | 18.5 | −7.6 | 2.7 |
| | | | 18.2 | | | |
| | | | 18.2 | | | |

ALQ: Above limit of quantitation

Interference from C5 was tested. Interference from C5 was not observed (Table 8).

TABLE 8

| | Spiked hC5 | Measured | |
|---|---|---|---|
| Nominal Anti-hC5 | concentration | Anti-hC5 Antibody | |
| Antibody concentration | in serum | concentration | RE |
| in serum (ng/mL) | (μg/mL) | in serum (ng/mL) | (%) |
| 2560 | 500 | 2405 | −6.1 |
| 2560 | 50.0 | 2396 | −6.4 |
| 2560 | 0 | 2376 | −7.2 |
| 400 | 500 | 403 | 0.8 |
| 400 | 50.0 | 419 | 4.8 |
| 400 | 0 | 398 | −0.5 |
| 100 | 500 | 102 | 1.7 |
| 100 | 50.0 | 102 | 2.4 |
| 100 | 0 | 95.0 | −5.0 |

A method to measure an Fc-Mutated Antibody in human serum using an Anti Fc-Mutation Antibody was established. The scheme of this assay is illustrated in FIG. 3.

### Example 5

We also tried to establish an assay for detecting an antigen that is recognized by an Fc-Mutated Antibody in a biological sample. Anti-hC5 Antibody was also used as a model of Fc-Mutated Antibody in this evaluation.

### Evaluation of the Method to Measure C5 in Human Serum (Antigen Detection Assay) Assay Procedures

Ninety six well immunoplate was coated with rabbit ant-hC5 monoclonal antibody and blocked with blocking buffer. Diluted serum samples were added to each well of the plate. Anti-hC5 Antibody was added to each well of the plate. The mouse Anti Fc-Mutation Antibody which was obtained by substitution of the Fc region in SKA0141 to mouse Fc region was added to each well of the plate followed by the addition of anti-mouse-POD (Jackson ImmunoResearch Inc.). Finally, POD substrate was added to each well of the plate and OD was measured. The plate was washed between the steps.

### Method Evaluation

Reproducibility was tested. Intra-batch accuracy (RE) and precision (CV) were −8.2% to 4.2% and 2.3% to 6.2% respectively (Table 9). Inter-batch accuracy (RE) and precision (CV) were −6.8% to 1.3% and 3.5% to 6.2% respectively (Table 10).

TABLE 9
Measured concentration in serum (μg/mL)

| | 3.25 μg/mL* | 6.50 μg/mL* | 89.1 μg/mL† | 154 μg/mL‡ | 208 μg/mL* |
|---|---|---|---|---|---|
| | 3.10 | 6.31 | 97.1 | 171 | 198 |
| | 3.45 | 6.53 | 95.1 | 152 | 196 |
| | 3.10 | 6.36 | 92.3 | 153 | 193 |
| | 3.31 | 6.36 | 91.1 | 149 | 188 |
| | 3.10 | 6.25 | 91.5 | 148 | 187 |
| | 2.96 | 6.08 | 89.7 | 144 | 185 |
| Mean | 3.17 | 6.32 | 92.8 | 153 | 191 |
| CV (%) | 5.6 | 2.3 | 3 | 6.2 | 2.8 |
| RE (%) | −2.5 | −2.8 | 4.2 | −0.6 | −8.2 |
| n | 6 | 6 | 6 | 6 | 6 |

*Recombinant human C5 was spiked in C5 depleted serum.
†Pooled human serum (endogenous human C5). The concentration was mean measured concentration in inter batch reproducibility test.
‡Recombinant human C5 was spiked in pooled human serum. The concentration was endogenous human C5 concentration (89.1 μg/mL) + spiked recombinant human C5 concentration (65.0 μg/mL).

TABLE 10
Measured concentration in serum (μg/mL)

| Assay run | 3.25 μg/mL* | 6.50 μg/mL* | 89.1 μg/mL† | 154 μg/mL‡ | 208 μg/mL* |
|---|---|---|---|---|---|
| 1 | 3.10 | 6.31 | 97.1 | 171 | 198 |
| | 3.45 | 6.53 | 95.1 | 152 | 196 |
| | 3.10 | 6.36 | 92.3 | 153 | 193 |
| | 3.31 | 6.36 | 91.1 | 149 | 188 |
| | 3.10 | 6.25 | 91.5 | 148 | 187 |
| | 2.96 | 6.08 | 89.7 | 144 | 185 |
| 2 | 3.07 | 6.17 | 87.7 | 160 | 206 |
| | 3.15 | 6.23 | 88.9 | 161 | 205 |
| | 2.84 | 5.92 | 86.6 | 159 | 203 |
| | 2.92 | 5.85 | 81.8 | 157 | 206 |
| | 3.31 | 5.92 | 85.8 | 151 | 196 |
| | 2.84 | 6.11 | 83.7 | 155 | 201 |
| 3 | 2.86 | 6.72 | 91.2 | 159 | 201 |
| | 3.08 | 6.2 | 90.9 | 157 | 206 |
| | 2.86 | 6.14 | 89.6 | 157 | 197 |
| | 2.86 | 6.03 | 86.7 | 160 | 208 |
| | 2.86 | 5.85 | 87.2 | 159 | 196 |
| | 2.86 | 5.73 | 87.6 | 156 | 201 |
| Mean | 3.03 | 6.15 | 89.1 | 156 | 199 |
| CV (%) | 6.20 | 4.1 | 4.2 | 3.9 | 3.5 |
| RE (%) | −6.8 | −5.4 | — | 1.3 | −4.3 |
| n | 18 | 18 | 18 | 18 | 18 |

*Recombinant human C5 was spiked in C5 depleted serum.
†Pooled human serum (endogenous human C5). The concentration was mean measured concentration in inter batch reproducibility test.
‡Recombinant human C5 was spiked in pooled human serum. The concentration was endogenous human C5 concentration (89.1 μg/mL) + spiked recombinant human C5 concentration (65.0 μg/mL).

Parallelism was tested. Ten individual sera were serially diluted from 325-fold to 2600-fold and measured. In any dilution factors, measured concentrations were recovered (Table 11).

TABLE 11

| | | Measured |
|---|---|---|
| Individual | | Concentration |
| human | Dilution | in serum |
| serum No. | factor | (μg/mL) |
| 1 | 325 | 73.3 |
| | 650 | 75.1 |
| | 1300 | 74.3 |
| | 2600 | 74.7 |
| 2 | 325 | 88.5 |
| | 650 | 90.5 |
| | 1300 | 92.0 |
| | 2600 | 90.4 |
| 3 | 325 | 59.1 |
| | 650 | 57.7 |
| | 1300 | 57.9 |
| | 2600 | 59.4 |
| 4 | 325 | 86.2 |
| | 650 | 89.3 |
| | 1300 | 88.1 |
| | 2600 | 89.6 |
| 5 | 325 | 76.5 |
| | 650 | 77.6 |
| | 1300 | 76.7 |
| | 2600 | 76.8 |
| 6 | 325 | 78.2 |
| | 650 | 83.0 |
| | 1300 | 84.3 |
| | 2600 | 84.0 |
| 7 | 325 | 56.2 |
| | 650 | 56.0 |
| | 1300 | 54.5 |
| | 2600 | 54.2 |
| 8 | 325 | 63.9 |
| | 650 | 65.8 |
| | 1300 | 66.1 |
| | 2600 | 66.4 |
| 9 | 325 | 65.2 |
| | 650 | 64.8 |
| | 1300 | 65.0 |
| | 2600 | 66.7 |
| 10 | 325 | 89.4 |
| | 650 | 94.3 |
| | 1300 | 95.5 |
| | 2600 | 96.7 |

Dilution linearity was tested. One thousand one hundred and thirty microgram per milliliter of C5 could be measured in dilution factor of 26,000-fold and prozone effect was not observed. (Table 12).

TABLE 12

| Nominal | | Measured | Mean measured | |
|---|---|---|---|---|
| Concentration | | concentration in | concentration in | |
| in serum | Dilution | diluted sample | serum sapmple | RE |
| (μg/mL) | Factor | (μg/mL) | (μg/mL) | (%) |
| 1130 | 650 | ALQ | — | — |
| | | ALQ | — | — |
| | | ALQ | — | — |
| 1130 | 2080 | ALQ | — | — |
| | | ALQ | — | — |
| | | ALQ | — | — |
| 1130 | 26,000 | 0.0439 | 1140 | 0.9 |
| | | 0.0421 | 1090 | −3.5 |
| | | 0.0414 | 1080 | −4.4 |

ALQ: Above limit of quantitation

Interference from Anti-hC5 Antibody was tested. Interference from Anti-hC5 Antibody was not observed (Table 13).

TABLE 13

| | Spiked Anti−hC5 | Measured C5 | |
|---|---|---|---|
| hC5 nominal | Antibody | concentration | |
| concentration | concentration | in serum | RE |
| (μg/mL) | (μg/mL) | (μg/mL) | (%) |
| 6.5 | 0 | 6.14 | −5.5 |
| | 6.50 | 5.41 | −16.8 |
| | 65.0 | 5.57 | −14.3 |
| | 650 | 5.80 | −10.8 |
| 87.3 | 0 | 91.3 | 4.6 |
| | 6.50 | 92.6 | 6.1 |
| | 65.0 | 93.7 | 7.3 |
| | 650 | 104 | 19.1 |
| 154 | 0 | 149 | −3.2 |
| | 6.50 | 150 | −2.6 |
| | 65.0 | 153 | −0.6 |
| | 650 | 167 | 8.4 |

A method to measure an antigen in human serum using an Anti Fc-Mutation Antibody was established. The scheme of this assay is illustrated in FIG. 4.

### Example 6

### Evaluation of the Method to Measure Anti-IL-8 Antibody in Human Plasma (Fc-Mutated Antibody Detection Assay)

### Assay Procedures

A specific anti-human IL-8 antibody that comprises a modified IgG heavy chain constant region comprising part of the mutations in the Fc region of SG115 (SEQ ID NO: 110) was used as a model of Fc-Mutated Antibody in EXAMPLEs 6-8, which is hereinafter referred to as “Anti-IL-8 Antibody”.

Ninety six well immunoplate was coated with one of rabbit Anti Fc-Mutation Antibodies (SKA0117) and blocked with blocking buffer. Diluted plasma samples were added to each well of the plate. Recombinant human IL-8 (SEQ ID NO: 111) was added to each well of the plate. Mouse anti-IL-8 monoclonal antibody (a heavy chain variable region, SEQ ID NO: 112; a light chain variable region, SEQ ID NO: 113; a heavy chain constant region, SEQ ID NO: 114; a light chain constant region, SEQ ID NO: 115) was added followed by the addition of anti-mouse-POD (Jackson ImmunoResearch Inc.). Finally, POD substrate was added to each well of the plate and OD was measured. The plate was washed between the steps.

### Method Evaluation

Reproducibility was tested. Anti-IL-8 Antibody of known concentration (50.0 ng/ml (REP-LL), 100 ng/ml (REP-L), 400 ng/ml (REP-M), 2400 ng/ml (REP-H), and 3200 ng/ml (REP-UL)) was measured. Intra-batch accuracy (RE) and precision (CV) were −14.2% to −9.7% and 4.9% to 7.3% respectively (Table 14).

TABLE 14

| | | REP-LL | | REP-L | | REP-M | | REP-H | | REP-UL | |
|---|---|---|---|---|---|---|---|---|---|---|---|
| | | 50.0 | | 100 | | 400 | | 2400 | | 3200 | |
| | Run ID | ng/mL | % RE | ng/mL | % RE | ng/mL | % RE | ng/mL | % RE | ng/mL | % RE |
| | 2 | 47.3 | −5.4 | 93.0 | −7.0 | 368 | −8.0 | 2340 | −2.5 | 3080 | −3.8 |
| | | 47.6 | −4.8 | 96.5 | −3.5 | 401 | 0.3 | 2100 | −12.5 | 2850 | −10.9 |
| | | 43.9 | −12.2 | 91.2 | −8.8 | 352 | −12.0 | 2040 | −15.0 | 2830 | −11.6 |
| | | 45.6 | −8.8 | 89.1 | −10.9 | 354 | −11.5 | 1950 | −18.8 | 2680 | −16.3 |
| | | 42.2 | −15.6 | 83.5 | −16.5 | 340 | −15.0 | 1990 | −17.1 | 2910 | −9.1 |
| | | 43.2 | −13.6 | 88.4 | −11.6 | 344 | −14.0 | 1930 | −19.6 | 2690 | −15.9 |
| Mean | | 45.0 | | 90.3 | | 360 | | 2060 | | 2840 | |
| % CV | | 4.9 | | 4.9 | | 6.2 | | 7.3 | | 5.2 | |
| % RE | | −10.0 | | −9.7 | | −10.0 | | −14.2 | | −11.3 | |
| % Total Error | | 14.9 | | 14.6 | | 16.2 | | 21.5 | | 16.5 | |
| n | | 6 | | 6 | | 6 | | 6 | | 6 | |

Inter-batch accuracy (RE) and precision (CV) were −10.3% to −6.6% and 7.0% to 10.1% respectively (Table 15).

TABLE 15

| | | REP-LL | | REP-L | | REP-M | | REP-H | | REP-UL | |
|---|---|---|---|---|---|---|---|---|---|---|---|
| | | 50.0 | | 100 | | 400 | | 2400 | | 3200 | |
| | Run ID | ng/mL | % RE | ng/mL | % RE | ng/mL | % RE | ng/mL | % RE | ng/mL | % RE |
| | 5 | 47.3 | −5.4 | 93.0 | −7.0 | 368 | −8.0 | 2340 | −2.5 | 3080 | −3.8 |
| | | 47.6 | −4.8 | 96.5 | −3.5 | 401 | 0.3 | 2100 | −12.5 | 2850 | −10.9 |
| | | 43.9 | −12.2 | 91.2 | −8.8 | 352 | −12.0 | 2040 | −15.0 | 2830 | −11.6 |
| | 3 | 42.3 | −15.4 | 89.9 | −10.1 | 359 | −10.3 | 2370 | −1.3 | 3190 | −0.3 |
| | | 41.1 | −17.8 | 90.5 | −9.5 | 361 | −9.8 | 2290 | −4.6 | 3090 | −3.4 |
| | | 42.3 | −15.4 | 91.4 | −8.6 | 367 | −8.3 | 2250 | −6.3 | 2980 | −6.9 |
| | 4 | 44.8 | −10.4 | 81.8 | −18.2 | 326 | −18.5 | 2450 | 2.1 | 3600 | 12.5 |
| | | 45.7 | −8.6 | 81.1 | −18.9 | 310 | −22.5 | 2160 | −10.0 | 2880 | −10.0 |
| | | 44.4 | −11.2 | 82.0 | −18.0 | 322 | −19.5 | 2310 | −3.8 | 2950 | −7.8 |
| | 6 | 49.8 | −0.4 | 92.8 | −7.2 | 379 | −5.3 | 2450 | 2.1 | 3310 | 3.4 |
| | | 49.2 | −1.6 | 96.9 | −3.1 | 385 | −3.8 | 2310 | −3.8 | 2870 | −10.3 |
| | | 48.5 | −3.0 | 96.6 | −3.4 | 399 | −0.3 | 2390 | −0.4 | 3080 | −3.8 |
| | 7 | 51.2 | 2.4 | 99.5 | −0.5 | 388 | −3.0 | 2290 | −4.6 | 3020 | −5.6 |
| | | 52.3 | 4.6 | 105 | 5.0 | 405 | 1.3 | 2310 | −3.8 | 2940 | −8.1 |
| | | 52.3 | 4.6 | 100 | 0.0 | 406 | 1.5 | 2140 | −10.8 | 2920 | −8.8 |
| | 8 | 43.3 | −13.4 | 84.1 | −15.9 | 316 | −21.0 | 2150 | −10.4 | 2960 | −7.5 |
| | | 44.2 | −11.6 | 79.5 | −20.5 | 299 | −25.3 | 1850 | −22.9 | 2690 | −15.9 |
| | | 44.2 | −11.6 | 82.3 | −17.7 | 313 | −21.8 | 2100 | −12.5 | 2600 | −18.8 |
| Mean | | 46.4 | | 90.8 | | 359 | | 2240 | | 2990 | |
| % CV | | 7.6 | | 8.3 | | 10.1 | | 7.0 | | 7.6 | |
| % RE | | −7.2 | | −9.2 | | −10.3 | | −6.7 | | −6.6 | |
| % Total Error | | 14.8 | | 17.5 | | 20.3 | | 13.6 | | 14.1 | |
| n | | 18 | | 18 | | 18 | | 18 | | 18 | |

Selectivity was tested. Without spiked Anti-IL-8 Antibody (SEL-O or SEL-EM-O), measured concentrations of all individual samples were BLQ. With spiked Anti-IL-8 Antibody (50.0 ng/ml (SEL-LL or SEL-EM-LL)), RE of measured concentrations of all individual samples were −23.2% to −4.3% (Table 16).

TABLE 16

| | | SEL-O or SEL-EM-O | | SEL-LL or SEL-EM-LL | |
|---|---|---|---|---|---|
| Run ID | Individual human plasma No. | 0.00 ng/mL | % RE | 50.0 ng/mL | % RE |
| 1 | No.1 | BLQ* | — | 46.5 | −7.0 |
| | No.2 | BLQ* | — | 42.6 | −14.8 |
| | No.3 | BLQ* | — | 42.6 | −14.8 |
| | No.4 | BLQ* | — | 43.6 | −12.8 |
| | No.5 | BLQ* | — | 40.7 | −18.6 |
| | No.6 | BLQ* | — | 44.1 | −11.8 |
| | No.7 | BLQ* | — | 47.0 | −6.0 |
| | No.8 | BLQ* | — | 47.6 | −4.8 |
| | No.9 | BLQ* | — | 43.4 | −13.2 |
| | No.10 | BLQ* | — | 43.1 | −13.8 |
| | No.1 of endometriosis donors | BLQ* | — | 39.9 | −20.2 |
| | No.2 of endometriosis donors | BLQ* | — | 38.4 | −23.2 |
| | No.3 of endometriosis donors | BLQ* | — | 39.4 | −21.2 |
| | No.4 of endometriosis donors | BLQ* | — | 44.9 | −10.2 |

*Below the lower limit of quantification

Dilution linearity was tested. One point six milligram per milliliter of Anti-IL-8 Antibody could be measured in dilution factor of 10,000-fold and prozone effect was not observed (Table 17).

TABLE 17

| | Conc. in | Dilution | Conc. in assay | Measured | | | |
|---|---|---|---|---|---|---|---|
| Sample | plasma | Factor (including | well | value | Mean | | |
| name | (ng/mL) | MRD) | (ng/mL) | (ng/mL) | (ng/mL) | % RE | % CV |
| DIL-10000 | 1,600,000 | 500,000 | 3.20 | 1,490,000 | 1,530,000 | −4.4 | 2.3 |
| | | | | 1,560,000 | | | |
| | | | | 1,530,000 | | | |
| DIL-1000 | 1,600,000 | 50,000 | 32.0 | 1,520,000 | 1,530,000 | −4.4 | 4.6 |
| | | | | 1,610,000 | | | |
| | | | | 1,470,000 | | | |
| DIL-100 | 1,600,000 | 5,000 | 320 | ALQ* | — | — | — |
| | | | | ALQ* | | | |
| | | | | ALQ* | | | |
| DIL-10 | 1,600,000 | 500 | 3200 | ALQ* | — | — | — |
| | | | | ALQ* | | | |
| | | | | ALQ* | | | |

Quantification range: 1.00 ng/mL ~64.0 ng/mL in assay well
*Above the upper limit of quantification

Interference from IL-8 was tested. IL-8 up to 50.0 ng/mL did not interfere with the assay at Anti-IL-8 Antibody concentration of 2400 ng/ml, and IL-8 up to 1.00 ng/ml did not interfere with the assay at Anti-IL-8 Antibody concentration of 50.0 ng/ml (Table 18).

TABLE 18

| | Anti-IL-8 | IL-8 | | | |
|---|---|---|---|---|---|
| | Antibody | conc. | | | |
| | conc. | in | Measured | | |
| Sample | in plasma | plasma | value | % | Mean |
| name | (ng/mL) | (ng/mL) | (ng/mL) | RE | (% RE) |
| IntH-H | 2400 | 200 | 3490 | 45.4 | 3570 (48.8) |
| | | | 3640 | 51.7 | |
| IntM-H | 2400 | 100 | 2990 | 24.6 | 2990 (24.6) |
| | | | 2980 | 24.2 | |
| IntL-H | 2400 | 50.0 | 2660 | 10.8 | 2610 (8.8) |
| | | | 2550 | 6.3 | |
| H-Cnt | 2400 | 0 | 2070 | −13.8 | 2060 (−14.2) |
| | | | 2040 | −15.0 | |
| IntH-LL | 50.0 | 10.0 | 70.4 | 40.8 | 72.6 (45.2) |
| | | | 74.8 | 49.6 | |
| IntM-LL | 50.0 | 5.00 | 64.0 | 28.0 | 64.7 (29.4) |
| | | | 65.3 | 30.6 | |
| IntL-LL | 50.0 | 1.00 | 56.6 | 13.2 | 55.9 (11.8) |
| | | | 55.2 | 10.4 | |
| LL-Cnt | 50.0 | 0 | 49.2 | −1.6 | 48.2 (−3.6) |
| | | | 47.2 | −5.6 | |

The effectiveness of the method to measure Anti-IL-8 Antibody in plasma was confirmed.

### Example 7

### Evaluation of the Method to Measure IL-8 in Human Plasma Using ELISA (Antigen Detection Assay)

### Assay Procedures

Ninety six well streptavidin immunoplate was coated with biotinylated mouse ant-IL-8 monoclonal antibody after blocked with blocking buffer. Anti-IL-8 Antibody was added to diluted plasma samples in 96 well polypropylene plate (reaction solution). After incubation, reaction solution was transferred to each well of the streptavidin plate. One of rabbit Anti Fc-Mutation Antibodies (SKA0001) was added followed by the addition of anti-rabbit-HRP (Southern Biotechnology Associates Inc.). Finally, POD substrate was added to each well of the plate and OD was measured. The streptavidin plate was washed between the steps.

### Method Evaluation

Reproducibility was tested. Intra-batch accuracy (RE) and precision (CV) were −6.4% to −1.9% and 1.5% to 2.9% respectively (Table 19).

TABLE 19

| | Spiked | Converted | | | |
|---|---|---|---|---|---|
| Sample | concentration | value | Mean | CV | RE |
| name | (pg/mL) | (pg/mL) | (pg/mL) | (%) | (%) |
| R5 | 100 | 91.6 | 93.6 | 1.5 | −6.4 |
| | | 93.7 | | | |
| | | 94.5 | | | |
| | | 95.0 | | | |
| | | 94.5 | | | |
| | | 92.2 | | | |
| R4 | 200 | 234 | 225 | 2.1 | — |
| | | 223 | | | |
| | | 220 | | | |
| | | 224 | | | |
| | | 224 | | | |
| | | 227 | | | |
| R3 | 400 | 420 | 429 | 2.5 | — |
| | | 424 | | | |
| | | 449 | | | |
| | | 424 | | | |
| | | 426 | | | |
| | | 434 | | | |
| R2 | 2560 | 2460 | 2470 | 1.5 | — |
| | | 2490 | | | |
| | | 2510 | | | |
| | | 2460 | | | |
| | | 2510 | | | |
| | | 2420 | | | |
| R1 | 3200 | 3120 | 3140 | 2.9 | −1.9 |
| | | 3250 | | | |
| | | 3170 | | | |
| | | 3140 | | | |
| | | 3180 | | | |
| | | 2980 | | | |

Converted values out of the quantitation range (below 100 pg/mL in plasma, above 3200 pg/mL plasma) were included in the calculation.
The result of intra-day reproducibility was used as the result of inter-day reproducibility 1st Assay.
CV: Coefficient of variation
RE: Relative error

Inter-batch accuracy (RE) and precision (CV) were −8.0% to 2.1% and 3.0% to 5.2% respectively (Table 20).

TABLE 20

| | Sample name | | | | | | | | | | | | | | | | |
|---|---|---|---|---|---|---|---|---|---|---|---|---|---|---|---|---|---|
| | R5 | | | | R4 | | | R3 | | | R2 | | | R1 | | | |
| | Spiked concentration (pg/mL) | | | | | | | | | | | | | | | | |
| | 100 | | | | 200 | | | 400 | | | 2560 | | | 3200 | | | |
| | Con- | | | | Con- | | | Con- | | | Con- | | | Con- | | | |
| | verted | Mean | | | verted | Mean | | verted | Mean | | verted | Mean | | verted | Mean | | |
| Batch | value | (pg/ | CV | RE | value | (pg/ | CV | value | (pg/ | CV | value | (pg/ | CV | value | (pg/ | CV | RE |
| No. | (pg/mL) | mL) | (%) | (%) | (pg/mL) | mL) | (%) | (pg/mL) | mL) | (%) | (pg/mL) | mL) | (%) | (pg/mL) | mL) | (%) | (%) |
| 190619 | 91.6 | 93.6 | 1.5 | −6.4 | 234 | 225 | 2.1 | 420 | 429 | 2.5 | 2460 | 2470 | 1.5 | 3120 | 3140 | 2.9 | −1.9 |
| (1st | 93.7 | | | | 223 | | | 424 | | | 2490 | | | 3250 | | | |
| run) | 94.5 | | | | 220 | | | 449 | | | 2510 | | | 3170 | | | |
| | 95.0 | | | | 224 | | | 424 | | | 2460 | | | 3140 | | | |
| | 94.5 | | | | 224 | | | 426 | | | 2510 | | | 3180 | | | |
| | 92.2 | | | | 227 | | | 434 | | | 2420 | | | 2980 | | | |
| 190620 | 90.8 | 92.5 | 3.7 | −7.5 | 233 | 235 | 2.1 | 435 | 418 | 3.4 | 2660 | 2610 | 2.6 | 3080 | 3290 | 5.7 | 2.7 |
| (2nd | 93.5 | | | | 229 | | | 421 | | | 2650 | | | 3360 | | | |
| run) | 94.1 | | | | 238 | | | 415 | | | 2620 | | | 3020 | | | |
| | 94.1 | | | | 233 | | | 394 | | | 2580 | | | 3390 | | | |
| | 96.1 | | | | 235 | | | 416 | | | 2660 | | | 3460 | | | |
| | 86.5 | | | | 244 | | | 429 | | | 2480 | | | 3410 | | | |
| 190621 | 87.1 | 89.9 | 2.1 | −10.1 | 237 | 226 | 3.3 | 428 | 414 | 3.6 | 2440 | 2520 | 4.2 | 3140 | 3370 | 4.1 | 5.4 |
| (3rd | 91.7 | | | | 224 | | | 411 | | | 2390 | | | 3340 | | | |
| run) | 88.6 | | | | 225 | | | 406 | | | 2590 | | | 3440 | | | |
| | 90.2 | | | | 231 | | | 391 | | | 2600 | | | 3470 | | | |
| | 89.6 | | | | 216 | | | 416 | | | 2640 | | | 3530 | | | |
| | 92.2 | | | | 224 | | | 432 | | | 2460 | | | 3310 | | | |
| n = 18 | — | 92.0 | 3.0 | −8.0 | — | 229 | 3.1 | — | 421 | 3.4 | — | 2530 | 3.6 | — | 3270 | 5.2 | 2.1 |

Converted values out of the quantitation range (below 100 pg/mL in plasma, above 3200 pg/mL in plasma) were included in the calculation.
The result of intra-day reproducibility was used as the result of inter-day reproducibility 1st Assay.
CV: Coefficient of variation
RE: Relative error

Dilution linearity was tested. One microgram per milliliter of IL-8 (spiked) could be measured in dilution factor 20,000-fold and prozone effect was not observed (Table 21).

TABLE 21
Spiked concentration in pooled human plasma: 1000 ng/mL

| | | Measured | Converted | | | |
|---|---|---|---|---|---|---|
| Sample | Dilution | value | value | Mean | CV | RE |
| name | factor | (pg/mL) | (pg/mL) | (pg/mL) | (%) | (%) |
| DL1 | 5 | ALQ | — | — | — | — |
| | | ALQ | — | | | |
| | | ALQ | — | | | |
| DL2 | 400 | ALQ | — | — | — | — |
| | | ALQ | — | | | |
| | | ALQ | — | | | |
| DL3 | 2500 | 368 | 921,000 | 954,000 | 3.1 | −4.6 |
| | | 385 | 961,000 | | | |
| | | 391 | 978,000 | | | |
| DL4 | 20,000 | 47.7 | 954,000 | 948,000 | 0.8 | −5.2 |
| | | 47.0 | 939,000 | | | |
| | | 47.6 | 951,000 | | | |

ALQ: Above upper limit of quantitation (>640 pg/mL)
CV: Coefficient of variation
RE: Relative error

Interference from Anti-IL-8 Antibody was tested. Interference from Anti-IL-8 Antibody (100 micro-g/mL in plasma) was not observed (Table 22).

TABLE 22
Batch No.190702

| | IL-8 spiked | Anti-IL-8 Antibody | Anti Fc-Mutation | | |
|---|---|---|---|---|---|
| | concentration | spiked concentration | Antibody spiked | Converted | |
| Sample | in plasma | in plasma | concentration in | value | % |
| name | (pg/mL) | (μg/mL) | plasma (μg/mL) | (pg/mL) | difference |
| IS1 | 400 | 100 | 100 | 2040 | 381.6 |
| IS2 | 400 | 100 | 10.0 | 628 | 48.2 |
| IS3 | 400 | 100 | 1.00 | 449 | 5.9 |
| IS4 | 400 | 100 | 0 | 428 | 0.8 |
| IS5 | 400 | 10.0 | 100 | 2990 | 606.4 |
| IS6 | 400 | 10.0 | 10.0 | 632 | 49.1 |
| IS7 | 400 | 10.0 | 1.00 | 452 | 6.6 |
| IS8 | 400 | 10.0 | 0 | 432 | 1.8 |
| IS9 | 400 | 0 | 100 | 2060 | 386.8 |
| IS10 | 400 | 0 | 10.0 | 618 | 45.9 |
| IS11 | 400 | 0 | 1.00 | 449 | 5.8 |
| IS12 | 400 | 0 | 0 | 424 | NA |
| IS13 | 100 | 100 | 100 | 1610 | 1358.5 |
| IS14 | 100 | 100 | 10.0 | 301 | 171.8 |
| IS15 | 100 | 100 | 1.00 | 132 | 19.5 |
| IS16 | 100 | 100 | 0 | 113 | 2.1 |
| IS17 | 100 | 10.0 | 100 | 1840 | 1563.8 |
| IS18 | 100 | 10.0 | 10.0 | 291 | 162.7 |
| IS19 | 100 | 10.0 | 1.00 | 133 | 20.0 |
| IS20 | 100 | 10.0 | 0 | 114 | 3.2 |
| IS21 | 100 | 0 | 100 | 1680 | 1419.5 |
| IS22 | 100 | 0 | 10.0 | 303 | 174.3 |
| IS23 | 100 | 0 | 1.00 | 131 | 18.1 |
| IS24 | 100 | 0 | 0 | 111 | NA |
| IS25 | 0 | 100 | 100 | 1480 | NA |
| IS26 | 0 | 100 | 10.0 | 182 | NA |
| IS27 | 0 | 100 | 1.00 | 21.6 | NA |
| IS28 | 0 | 100 | 0 | 1.63 | NA |
| IS29 | 0 | 10.0 | 100 | 1620 | NA |
| IS30 | 0 | 10.0 | 10.0 | 198 | NA |
| IS31 | 0 | 10.0 | 1.00 | 17.2 | NA |
| IS32 | 0 | 10.0 | 0 | ND | NA |
| IS33 | 0 | 0 | 100 | 1440 | NA |
| IS34 | 0 | 0 | 10.0 | 189 | NA |
| IS35 | 0 | 0 | 1.00 | 18.4 | NA |
| IS36 | 0 | 0 | 0 | ND | NA |

% difference = (converted value of IS added sample − converted value of IS free sample)/converted value of IS free sample × 100
IS: Interference substance
ND: Not detected
Underline: Below lower limit of quantitation (<100 pg/mL in plasma)
NA: Not applicable

The effectiveness of the method to measure IL-8 in human plasma using ELISA was confirmed.

### Example 8

### Evaluation of the Method to Measure IL-8 in Human Plasma Using Simoa (Registered Trademark) (Simoa (Registered Trademark) Assay)

### Assay Procedures

Assay was conducted automatically by using Simoa (registered trademark) system (Quanterix Corporation). Diluted samples, Anti-IL-8 Antibody, and mouse anti-IL-8 monoclonal antibody coated on beads were mixed. The beads were loaded into microwell of an array disk. Biotinylated Anti Fc-Mutation Antibody (SKA0028) was added to the disk followed by the addition of streptavidin-beta-galactosidase, SBG (Quanterix Corporation). Finally, substrate of beta-galactocidases, RGB, was added and fluorescent intensity was measured.

### Method Evaluation

Reproducibility was tested. Intra-batch precision (CV) were 1.3% to 14.3% (Table 23).

TABLE 23

| Plasma | | | | | | | |
|---|---|---|---|---|---|---|---|
| batch No. | PLA021B020C009 | PLA021A020C011 | PLA022C100E001 | PLA022A100E001 | PLA021A050000 | B061631P | B061633P |
| Run ID | 1 | 1 | 3 | 3 | 1 | 1 | 1 |
| Measured | 0.0734 | 0.0760 | 0.147 | 0.322 | 1.04 | 2.56 | 3.92 |
| value | 0.102 | 0.0982 | 0.156 | 0.324 | 1.07 | 2.57 | 3.78 |
| (pg/mL) | 0.0910 | 0.0854 | 0.147 | 0.344 | 1.16 | 2.54 | 4.14 |
| | 0.100 | 0.114 | 0.154 | 0.322 | 1.19 | 2.51 | 4.06 |
| | 0.0778 | 0.0864 | 0.143 | 0.270 | 1.13 | 2.53 | 3.97 |
| | 0.0925 | 0.0950 | 0.145 | 0.307 | 1.10 | 2.60 | 4.07 |
| % CV | 13.0 | 14.3 | 3.6 | 7.9 | 4.9 | 1.3 | 3.2 |
| n | 6 | 6 | 6 | 6 | 6 | 6 | 6 |

Inter-batch precision (CV) were 8.6% to 24.7% respectively (Table 24).

TABLE 24-1

| | PLA021B020C009 | | PLA021A020C011 | | PLA022C100E001 | | PLA022A100E001 | |
|---|---|---|---|---|---|---|---|---|
| | | Measured | | Measured | | Measured | | Measured |
| Plasma | Run | value | Run | value | Run | value | Run | value |
| batch No. | ID | (pg/mL) | ID | (pg/mL) | ID | (pg/mL) | ID | (pg/mL) |
| | 1 | 0.0734 | 1 | 0.0760 | 3 | 0.147 | 3 | 0.322 |
| | | 0.102 | | 0.0982 | | 0.156 | | 0.324 |
| | | 0.0910 | | 0.0854 | | 0.147 | | 0.344 |
| | 4 | 0.0577 | 4 | 0.0465 | 4 | 0.124 | 4 | 0.232 |
| | | 0.0702 | | 0.0421 | | 0.119 | | 0.251 |
| | | 0.0445 | | 0.0743 | | 0.147 | | 0.264 |
| | 5 | 0.0459 | 6 | 0.0844 | 5 | 0.148 | 5 | 0.315 |
| | | 0.0614 | | 0.0663 | | 0.131 | | 0.285 |
| | | 0.0687 | | 0.0683 | | 0.128 | | 0.283 |
| | 7 | 0.0766 | 7 | 0.0543 | 7 | 0.133 | 7 | 0.305 |
| | | 0.0712 | | 0.0534 | | 0.152 | | 0.317 |
| | | 0.0737 | | 0.0716 | | 0.136 | | 0.353 |

| | | | | |
|---|---|---|---|---|
| Mean Measured | 0.0697 | 0.0684 | 0.139 | 0.300 |
| value (pg/mL) | | | | |
| % CV | 23.7 | 24.7 | 8.6 | 12.4 |
| n | 12 | 12 | 12 | 12 |
| Dilution factor | 20 | 20 | 20 | 20 |
| Endogenous IL-8 | 1.39 | 1.37 | 2.78 | 5.99 |
| concentration in | | | | |
| plasma (pg/mL) | | | | |

TABLE 24-2

| | PL021A050000 | | B061631P | | B061633P | |
|---|---|---|---|---|---|---|
| | | Measured | | Measured | | Measured |
| | Run | value | Run | value | Run | value |
| Plasma batch No. | ID | (pg/mL) | ID | (pg/mL) | ID | (pg/mL) |
| | 1 | 1.04 | 1 | 2.56 | 1 | 3.92 |
| | | 1.07 | | 2.57 | | 3.78 |
| | | 1.16 | | 2.54 | | 4.14 |
| | 4 | 0.933 | 4 | 2.13 | 4 | 3.59 |
| | | 0.943 | | 2.26 | | 3.35 |
| | | 1.00 | | 2.01 | | 3.46 |
| | 5 | 0.878 | 5 | 1.95 | 5 | 3.09 |
| | | 0.909 | | 1.95 | | 3.17 |
| | | 0.862 | | 1.94 | | 3.33 |
| | 7 | 1.00 | 7 | 2.19 | 7 | 3.54 |
| | | 1.06 | | 2.16 | | 3.41 |
| | | 0.99 | | 2.11 | | 2.95 |

| | | | |
|---|---|---|---|
| Mean Measured | 0.986 | 2.20 | 3.48 |
| value (pg/mL) | | | |
| % CV | 8.8 | 11.0 | 9.9 |
| n | 12 | 12 | 12 |
| Dilution factor | 20 | 20 | 20 |
| Endogenous IL-8 | 19.7 | 44.0 | 69.6 |
| concentration in | | | |
| plasma (pg/mL) | | | |

Parallelism was tested. Three individual plasmas were serially diluted from 20-fold to 40-fold and measured. In any dilution factors, measured concentrations were recovered (Table 25).

TABLE 25

| | Measured | | Endogenous IL-8 | |
|---|---|---|---|---|
| | value | Dilution | concentration in plasma | % |
| Plasma batch No. | (pg/mL) | factor | (pg/mL) | Diff |
| PLA021A010BB228 | 0.423 | 20 | 8.46 | 0.0 |
| | 0.285 | 30 | 8.56 | 1.2 |
| | 0.203 | 40 | 8.11 | −4.2 |
| PLA021A010D012 | 0.757 | 20 | 15.1 | 0.0 |
| | 0.543 | 30 | 16.3 | 7.8 |
| | 0.432 | 40 | 17.3 | 14.2 |
| PLA021A050000 | 0.332 | 20 | 6.64 | 0.0 |
| | 0.215 | 30 | 6.46 | −2.8 |
| | 0.202 | 40 | 8.06 | 21.4 |

Dilution linearity was tested. Three point four eight microgram per milliliter of IL-8 could be measured in dilution factor 50,000-fold and prozone effect was not observed. (Table 26).

TABLE 26

| | | | IL-8 | Mean IL-8 | | | |
|---|---|---|---|---|---|---|---|
| | | | concentration | concentration | Theoretical | | |
| Sample | Measured value | Dilution | in plasma | in plasma | concentration | | |
| name | (pg/mL) | factor | (pg/mL) | (pg/mL) | (pg/mL) | % RE | % CV |
| DIL-50 | ALQ | 50 | — | — | | — | — |
| | ALQ | | — | — | | | |
| | ALQ | | — | — | | | |
| DIL-25,000 | 3.92 | 25,000 | 97,963.49 | 97444.76 | | −2.6 | 0.9 |
| | 3.86 | | 96,424.67 | | 100,005.99* | | |
| | 3.92 | | 97,946.10 | | | | |
| DIL-50,000 | 1.89 | 50,000 | 94,466.88 | 98160.95 | | −1.8 | 3.8 |
| | 1.96 | | 98,008.77 | | | | |
| | 2.04 | | 10,2007.21 | | | | |

—: Not applicable
*: Theoretical concentration was calculated considering concentration of the endogenous IL-8 in the plasma (batch No. PLA022A100E001) determined in between-run precision.

Interference from Anti-IL-8 Antibody was tested. Interference from Anti-IL-8 Antibody (100 micro-g/mL in plasma) was not observed (Table 27).

TABLE 27

| | Anti-IL-8 | | | Endogenous | |
|---|---|---|---|---|---|
| | Antibody | | | IL-8 | |
| | conc. in | Measured | | concentration | |
| Sample | plasma | value | Dilution | in plasma | % |
| name | (μg/mL) | (pg/mL) | factor | (pg/mL) | Int |
| INTO | 0 | 0.358 | 20 | 7.17 | 0.0 |
| INT1 | 1.00 | 0.353 | | 7.06 | −1.6 |
| INT2 | 10.0 | 0.359 | | 7.19 | 0.3 |
| INT3 | 30.0 | 0.371 | | 7.42 | 3.4 |
| INT4 | 60.0 | 0.364 | | 7.28 | 1.5 |
| INTS | 100 | 0.368 | | 7.36 | 2.7 |

The effectiveness of the method to measure IL-8 in human plasma using Simoa (registered trademark) assay was confirmed.

### Example 9

Evaluation of Affinity of Anti Fc-Mutation Antibodies that Show Selective Binding to SG115v1 Against Anti-hC5 Antibody.

K_{D} value of Anti Fc-Mutation Antibodies that show selective binding to SG115v1 (SKA0009, SKA0016, SKA0046, SKA0052, SKA0054, and SKA0127) against Anti-hC5 Antibody at pH 7.4 was determined at 25 degrees C. using Biacore T200 instrument (GE Healthcare).

Mouse anti-rabbit IgG (Fc) antibody (hereinafter called anti-rabbit IgG) (Abbexa) was immobilized onto flow cell (FC) 1 and 2 of a CM5 sensor chip using an amine coupling kit (GE Healthcare). For the immobilization of anti-rabbit IgG, HBS-EP+, pH7.4 (GE Healthcare) buffer was used as running buffer. After the immobilization, running buffer was changed to phosphate pH7.4 buffer (50 mM phosphate buffer containing 150 mM NaCl and 0.05 w/v % P-20, pH 7.4). Each Anti Fc-Mutation Antibody was captured onto FC2 of the sensor chip by anti-rabbit IgG. The amount of Anti Fc-Mutation Antibody to be captured was adjusted so that the number of resonance units (RU) was 100. Anti-hC5 Antibody was injected at 0, 50, 100, 200, 400, and 800 nM at 10 micro L/min. Sensor surface was regenerated after each cycle with 10 mM Glycine-HCl, pH2.0, which was injected at a flow rate of 30 micro L/min. K_{D} values were obtained using Biacore T200 Evaluation software, version 2.0 (GE Healthcare). Association rate (ka), dissociation rate (kd), and dissociation constant (K_{D}) are shown in Table 28.

TABLE 28

| Ligand | ka (1/Ms) | kd (1/s) | K_{D} (M) |
|---|---|---|---|
| SKA0009 | 7.69 × 10⁴ | *1.41 × 10⁻⁶ | *1.84 × 10⁻¹¹ |
| SKA0016 | 3.09 × 10⁴ | *5.90 × 10⁻⁷ | *1.91 × 10⁻¹¹ |
| SKA0046 | 8.85 × 10⁴ | 5.17 × 10⁻⁵ | *5.85 × 10⁻¹⁰ |
| SKA0052 | 1.08 × 10⁵ | 1.96 × 10⁻⁴ | 1.81 × 10⁻⁹ |
| SKA0054 | 2.73 × 10⁴ | 1.97 × 10⁻⁴ | 7.22 × 10⁻⁹ |
| SKA0127 | 7.18 × 10⁴ | 2.54 × 10⁻⁴ | 3.54 × 10⁻⁹ |

*the data reliability might be low due to slow dissociation rate

### Example 10

Evaluation of Affinity of Anti Fc-Mutation Antibodies that Show Selective Binding to SG115v1 Against Anti-IL-8 Antibody.

To confirm the binding ability of Anti Fc-Mutation Antibodies that show selective binding to SG115v1, K_{D} values of Anti Fc-Mutation Antibodies against Anti-IL-8 Antibody at pH 7.4 were determined at 25 degrees C. using Biacore T200 instrument (GE Healthcare). Sequence of Fc region of Anti-IL-8 Antibody has high similarity to that of Anti-hC5 Antibody.

Anti-rabbit IgG (Abbexa) was immobilized onto FC1 and 2 of a CM5 sensor chip using an amine coupling kit (GE Healthcare). For the immobilization of anti-rabbit IgG, HBS-EP+, pH7.4 (GE Healthcare) buffer was used as running buffer. After the immobilization, running buffer was changed to Phosphate pH7.4 buffer (50 mM phosphate buffer containing 150 mM NaCl and 0.05 w/v % P-20, pH 7.4). Each Anti Fc-Mutation Antibody was captured onto FC2 of the sensor chip by anti-rabbit IgG. The amount of Anti Fc-Mutation Antibody to be captured was adjusted so that the number of resonance units (RU) was 100. Anti-IL-8 Antibody was injected at 0, 100, 400, and 800 nM at 10 micro L/min. Sensor surface was regenerated after each cycle with 10 mM Glycine-HCl, pH2.0, which was injected at a flow rate of 30 micro L/min. K_{D} values were obtained using Biacore T200 Evaluation software, version 2.0 (GE Healthcare).

ka, kd, and K_{D} are listed in Table 29. While the amino acid at position 239 according to the EU numbering system in Anti-hC5 Antibody was mutated from Ser to Lys, the amino acid at the corresponding position in Anti-IL-8 Antibody was not mutated. In such situation, Anti Fc-Mutation Antibodies could bind to Anti-IL-8 Antibody. It means that the two mutations in SG115v1 which both Anti-hC5 Antibody and Anti-IL-8 Antibody commonly have, i.e. L235R and G236R (both positions are according to the EU numbering system), are essential for selective binding of Anti Fc-Mutation Antibodies to SG115v1.

TABLE 29

| Ligand | ka (1/Ms) | kd (1/s) | K_{D} (M) |
|---|---|---|---|
| SKA0009 | 2.50 × 10⁴ | 9.04 × 10⁻⁴ | 3.62 × 10⁻⁸ |
| SKA0016 | 1.18 × 10⁴ | 7.10 × 10⁻⁴ | 6.00 × 10⁻⁸ |
| SKA0046 | 4.20 × 10⁴ | 2.09 × 10⁻³ | 4.96 × 10⁻⁸ |
| SKA0052 | 9.17 × 10⁴ | 4.31 × 10⁻³ | 4.70 × 10⁻⁸ |
| SKA0054 | 9.98 × 10³ | 9.66 × 10⁻⁴ | 9.68 × 10⁻⁸ |
| SKA0127 | 1.77 × 10⁴ | 8.04 × 10⁻⁴ | 4.54 × 10⁻⁸ |

*the data reliability might be low due to slow dissociation rate

### Example 11

Evaluation of Affinity of Anti Fc-Mutation Antibodies that Show Selective Binding to SG115v2 Against Anti-DENV E Protein Antibody

K_{D} value of Anti Fc-Mutation Antibodies that show selective binding to SG115v2 (SKA0001, SKA0027, SKA0028, SKA0117, SKA0141, and SKA0171) against an anti-DENV E protein antibody comprising a modified IgG heavy chain constant region (SEQ ID NO: 116) as Fc-Mutated Antibody at pH 7.4 was determined at 25 degrees C. using Biacore T200 instrument (GE Healthcare). Anti-rabbit IgG was immobilized onto FC 3 and 4 of a CM5 sensor chip using an amine coupling kit (GE Healthcare). For the immobilization of anti-rabbit IgG, HBS-EP+, pH7.4 (GE Healthcare) buffer was used as running buffer. After the immobilization, running buffer was changed to phosphate pH7.4 buffer. Each antibody was captured onto FC4 of the sensor chip by anti-rabbit IgG. The amount of Anti Fc-Mutation Antibody to be captured was adjusted so that the number of resonance units (RU) was 100. The anti-DENV E protein antibody was injected at 0, 12.5, 50, and 400 nM at 10 micro L/min. Sensor surface was regenerated each cycle with 10 mM Glycine-HCl, pH2.0, which was injected at a flow rate of 30 micro L/min. K_{D} values were obtained using Biacore T200 Evaluation software, version 2.0 (GE Healthcare). ka, kd, and K_{D} are shown in Table 30.

TABLE 30

| Ligand | ka (1/Ms) | kd (1/s) | K_{D} (M) |
|---|---|---|---|
| SKA0001 | 4.44 × 10⁴ | *1.23 × 10⁻⁷ | *2.78 × 10⁻¹² |
| SKA0027 | 1.90 × 10⁵ | 1.91 × 10⁻⁴ | 1.00 × 10⁻⁹ |
| SKA0028 | 1.16 × 10⁵ | 7.27 × 10⁻⁵ | *6.26 × 10⁻¹⁰ |
| SKA0117 | 1.26 × 10⁵ | *3.46 × 10⁻⁹ | *2.75 × 10⁻¹³ |
| SKA0141 | 2.67 × 10⁵ | 1.35 × 10⁻³ | 5.04 × 10⁻⁹ |
| SKA0171 | 1.02 × 10⁶ | 1.49 × 10⁻³ | 1.45 × 10⁻⁹ |

*the data reliability might be low due to slow dissociation rate

### Example 12

### Evaluation of Affinity of Anti-hC5 Antibody Against Human C5 Using SKA0016 and SKA0117 as Capture Molecules.

K_{D} value of Anti-hC5 Antibody against human C5 at pH 7.4 was determined at 37 degrees C. using Biacore T200 instrument (GE Healthcare). SKA0016 was immobilized onto FC1 and 2, and SKA0117 was immobilized onto FC3 and 4 of a CM5 sensor chip using an amine coupling kit (GE Healthcare). For the immobilization of SKA0016 and SKA0117, HBS-EP+, pH7.4 (GE Healthcare) buffer was used as running buffer. After the immobilization, running buffer was changed to phosphate pH7.4 buffer. Anti-hC5 Antibody was captured onto FC2 and FC4 of the sensor chip by SKA0016 and SKA0117. The amount of Anti-hC5 antibody to be captured was adjusted so that the number of resonance units (RU) was 35. Human C5 was injected at 0, 2, 4, 8, 16, and 32 nM at 10 micro L/min. Sensor surface was regenerated each cycle with 100 mM Glycine-HCl, pH2.0 followed by 25 mM NaOH, which were both injected at a flow rate of 30 micro L/min. K_{D} values were obtained using Biacore T200 Evaluation software, version 2.0 (GE Healthcare). ka, kd, and K_{D} are listed in Table 31.

TABLE 31

| Capture molecule | ka (1/Ms) | kd (1/s) | K_{D} (M) |
|---|---|---|---|
| SKA0016 | 6.33 × 10⁵ | 1.13 × 10⁻⁴ | 1.78 × 10⁻¹⁰ |
| SKA0117 | 6.51 × 10⁵ | 1.23 × 10⁻⁴ | 1.88 × 10⁻¹⁰ |

### Example 13

Qualitative Analysis of pH-Dependent Interaction of Anti-hC5 Antibody Against Human C5 Using SKA0016 and SKA0117 as Immobilizing Molecules.

pH-dependent interaction between Anti-hC5 Antibody and human C5 at pH7.4 and pH6.0 was evaluated at 37 degrees C. using Biacore T200 instrument (GE Healthcare). Anti-hC5 Antibody was captured onto FC2 and FC4 of CM5 chip that was prepared in EXAMPLE 12. The amount of Anti-hC5 antibody to be captured was adjusted so that the number of resonance units (RU) was 35. To confirm the association between Anti-hC5 Antibody and human C5 at pH7.4, 32 nM of human C5 was injected to all FC in phosphate pH 7.4 buffer. Then dissociation phase was monitored in phosphate pH 7.4 buffer or phosphate pH6.0 buffer (50 mM phosphate buffer containing 150 mM NaCl and 0.05 w/v % P-20, pH 6.0) as running buffer. After monitoring the dissociation phase, the sensor chip was regenerated by injecting 100 mM Gly-HCl, pH2.0 followed by 25 mM NaOH, which were both injected at a flow rate of 30 micro L/min. The pH-dependent interaction of Anti-hC5 Antibody to human C5 was analyzed by comparing the dissociation phases of sensorgrams at pH 7.4 and pH 6.0 using Biacore T200 Evaluation Software Version 2.0. Sensorgram on FC2 was subtracted by FC1 and sensorgram of FC4 was subtracted by FC3, and each sensorgram was normalized by adjusting human C5 binding response, which was at 5 seconds before the end of human C5 injection, to the value ‘100’.

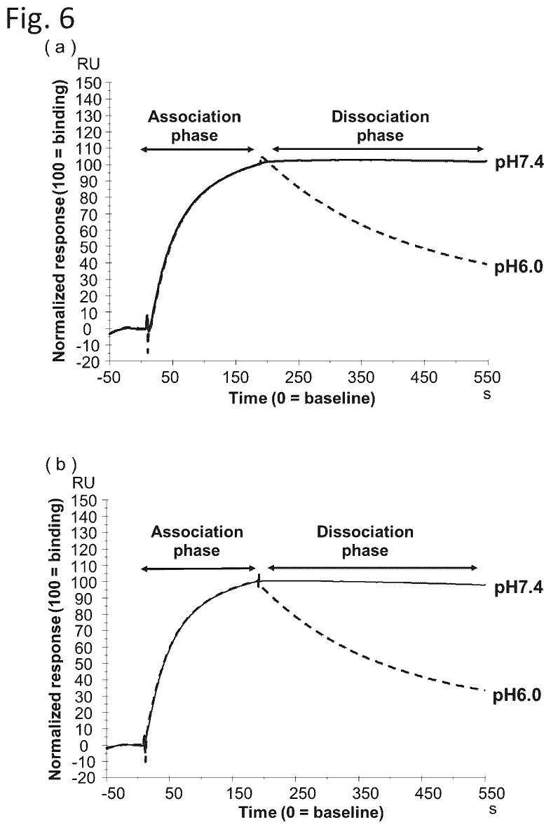

Regardless of the capture molecule, dissociation of human C5 from Anti-hC5 Antibody was more rapid on the condition with pH6.0 than with pH7.4 (FIG. 6; Anti-hC5 Antibody was immobilized by (a) SKA0016 and (b) SKA0117). Therefore, it is considered that both SKA0016 and SKA0117 are effective for monitoring pH-dependent interaction between Anti-hC5 Antibody and human C5.

### Example 14

Evaluation of Binding Between Human Fc Receptor (hFcRn) and Anti-hC5 Antibody that was Captured by SKA0016.

Whether human FcRn could bind to Anti-hC5 Antibody that was captured by SKA0016 at pH 6.0 was evaluated using Biacore T200 instrument (GE Healthcare). Anti-hC5 Antibody was captured onto FC2 by SKA0016 that was immobilized to CM5 chip by the same procedure as in EXAMPLE 9. Phosphate pH6.0 buffer was used as running buffer. The amount of Anti-hC5 antibody to be captured was adjusted so that the number of resonance units (RU) was 400. hFcRn was injected at 0, 26.3, 52.5, 105, 210, and 420 nM at 10 micro L/min in single cycle kinetics manner. Sensor surface was regenerated with 100 mM Glycine-HCl, pH2.0 followed by 25 mM NaOH, which were both injected at a flow rate of 30 micro L/min. Increment of binding response of hFcRn was confirmed using Biacore T200 Evaluation software, version 2.0 (GE Healthcare).

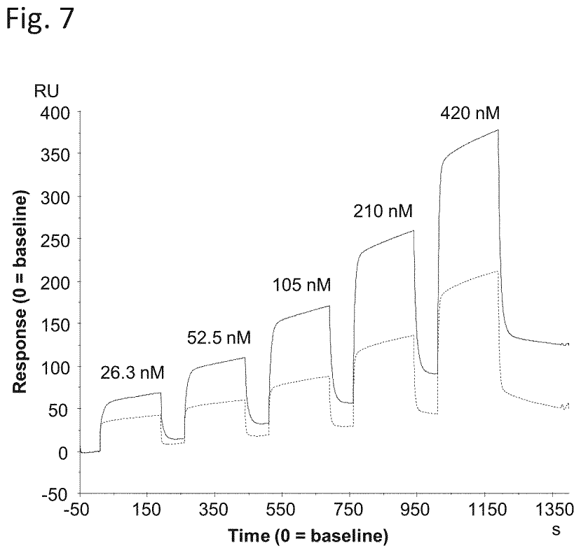

FIG. 7 shows sensorgram of FC1 (dash line) and FC2 (solid line). From the FC2 sensorgram (solid line), it is recognized that the binding response of hFcRn increases concentration-dependent manner. SKA0016 did not interrupt the binding between hFcRn and Anti-hC5 Antibody. However, hFcRn seems to bind to Fc region of SKA0016 because the sensorgram of FC1 also showed the increment of binding response (dash line). This undesirable human FcRn binding to capture molecule can be solved by introducing the amino acid substitutions which abrogate the binding to human FcRn into SKA0016.

1.-15. (canceled)

16. An isolated antibody which specifically binds to a modified human IgG heavy chain constant region, wherein the antibody comprises any one of following (a) to (f):
(a) variable regions that comprise
HVR-H1 comprising the amino acid sequence of SEQ ID NO: 33,
HVR-H2 comprising the amino acid sequence of SEQ ID NO: 45,
HVR-H3 comprising the amino acid sequence of SEQ ID NO: 57,
HVR-L1 comprising the amino acid sequence of SEQ ID NO: 69,
HVR-L2 comprising the amino acid sequence of SEQ ID NO: 81, and
HVR-L3 comprising the amino acid sequence of SEQ ID NO: 93;
(b) variable region that comprise
HVR-H1 comprising the amino acid sequence of SEQ ID NO: 34,
HVR-H2 comprising the amino acid sequence of SEQ ID NO: 46,
HVR-H3 comprising the amino acid sequence of SEQ ID NO: 58,
HVR-L1 comprising the amino acid sequence of SEQ ID NO: 70,
HVR-L2 comprising the amino acid sequence of SEQ ID NO: 82, and
HVR-L3 comprising the amino acid sequence of SEQ ID NO: 94;
(c) variable region that comprise
HVR-H1 comprising the amino acid sequence of SEQ ID NO: 37,
HVR-H2 comprising the amino acid sequence of SEQ ID NO: 49,
HVR-H3 comprising the amino acid sequence of SEQ ID NO: 61,
HVR-L1 comprising the amino acid sequence of SEQ ID NO: 73,
HVR-L2 comprising the amino acid sequence of SEQ ID NO: 85, and
HVR-L3 comprising the amino acid sequence of SEQ ID NO: 97;
(d) variable region that comprise
HVR-H1 comprising the amino acid sequence of SEQ ID NO: 38,
HVR-H2 comprising the amino acid sequence of SEQ ID NO: 50,
HVR-H3 comprising the amino acid sequence of SEQ ID NO: 62,
HVR-L1 comprising the amino acid sequence of SEQ ID NO: 74,
HVR-L2 comprising the amino acid sequence of SEQ ID NO: 86, and
HVR-L3 comprising the amino acid sequence of SEQ ID NO: 98;
(e) variable region that comprise
HVR-H1 comprising the amino acid sequence of SEQ ID NO: 39,
HVR-H2 comprising the amino acid sequence of SEQ ID NO: 51,
HVR-H3 comprising the amino acid sequence of SEQ ID NO: 63,
HVR-L1 comprising the amino acid sequence of SEQ ID NO: 75,
HVR-L2 comprising the amino acid sequence of SEQ ID NO: 87, and
HVR-L3 comprising the amino acid sequence of SEQ ID NO: 99; and
(f) variable region that comprise
HVR-H1 comprising the amino acid sequence of SEQ ID NO: 41,
HVR-H2 comprising the amino acid sequence of SEQ ID NO: 53,
HVR-H3 comprising the amino acid sequence of SEQ ID NO: 65,
HVR-L1 comprising the amino acid sequence of SEQ ID NO: 77,
HVR-L2 comprising the amino acid sequence of SEQ ID NO: 89, and
HVR-L3 comprising the amino acid sequence of SEQ ID NO: 101.

17. The antibody of claim 16, wherein the modified IgG heavy chain constant region is modified from an IgG1 constant region comprising the amino acid sequence of SEQ ID NO: 106, an IgG2 constant region comprising the amino acid sequence of SEQ ID NO: 107, an IgG3 constant region comprising the amino acid sequence of SEQ ID NO: 108, or an IgG4 constant region comprising the amino acid sequence of SEQ ID NO: 109.

18. The antibody of claim 16, wherein said modified IgG heavy chain constant region is derived from a chimeric constant region obtained from constant regions in human naturally occurring IgG1 and IgG4.

19. The antibody of claim 16, which binds to the modified IgG heavy chain constant region consisting of the amino acid sequence RRGPK (SEQ ID NO: 104).

20. An isolated nucleic acid encoding the antibody of claim 16.

21. A host cell comprising a vector comprising the nucleic acid of claim 20.

22. A method of making an antibody wherein the method comprises culturing the host cell of claim 21 under conditions suitable for expression of the antibody, and optionally recovering the antibody from the host cell or host cell culture medium.

23. The antibody of claim 16, wherein the modified IgG heavy chain constant region comprises at least one amino acid selected from Leu at position 428, Ala at position 434, Arg at position 438, and Glu at position 440 (all positions according to the EU numbering system).

24. The antibody of claim 16, wherein the modified IgG heavy chain constant region comprises Leu at position 428, Ala at position 434, Arg at position 438, and Glu at position 440 (all positions according to the EU numbering system).


SEQUENCE LISTING (118 sequences)

SEQ ID NO: 1 - protein, 328 aa, synthetic construct
  REGION 1..328: note = SG115
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 2 - protein, 328 aa, synthetic construct
  REGION 1..328: note = SG115v1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 3 - protein, 328 aa, synthetic construct
  REGION 1..328: note = SG115v2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 4 - protein, 328 aa, synthetic construct
  REGION 1..328: note = G1m
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 5 - protein, 325 aa, synthetic construct
  REGION 1..325: note = G4d
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 6 - protein, 323 aa, synthetic construct
  REGION 1..323: note = rbIgGv2
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPITHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 7 - protein, 104 aa, synthetic construct
  REGION 1..104: note = rbIgk
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 8 - protein, 114 aa, synthetic construct
  REGION 1..114: note = SKA0001-VH
QSVEESGGRLVTPGTPLTLTCTVSGFSLSYYAMNWVRQAPGKGLEWIGIIGISGGTYYASWAKGRFTISKSSSTTVDLKMTSPTTEDTATYFCARGGSAWGDLWGQGTLVTVSS

SEQ ID NO: 9 - protein, 115 aa, synthetic construct
  REGION 1..115: note = SKA0009-VH
QSVEESGGRLVTPGTSLTLTCTVSGIDLSSYGMGWVRQAPGMGLEYIGIIYAAGRTYYASWVKGRFIISKTSTTVDLEMTSLTTEDTATYFCARHGSWYAGMDLWGPGTLVTVSS

SEQ ID NO: 10 - protein, 124 aa, synthetic construct
  REGION 1..124: note = SKA0016-VH
QEQLVESGGGLVQPEGSLTLTCTASGFSFSSSDYMCWVRQAPGKGLEWIACIYIGSNGDTFYASWAKGRFTISKTSSTTVTLQMTSLTAADTATYFCARDSRRADDYGTRLDLWGQGTLVTVSS

SEQ ID NO: 11 - protein, 121 aa, synthetic construct
  REGION 1..121: note = SKA0027-VH
QEQLVESGGGLVQPEGSLTLTCTASGFSFSSSDYMCWVRQAPGKGLEWIGCIYTGSGSTYYASWAKGRFTISKTSSTTVTLQMTSLTAADTATFFCARRGDSYSNYSFKLWGPGTLVTVSS

SEQ ID NO: 12 - protein, 119 aa, synthetic construct
  REGION 1..119: note = SKA0028-VH
QSVEESGGRLVTPGTPLTLTCTASGFTISSYHMSWVRQAPGKGLEWIGIIHGSGNMWYASWAKGRFTISRTSTTVDLEITSPTTEDTATYFCARIRFWASSNYYYFYLWGQGTLVTVSS

SEQ ID NO: 13 - protein, 128 aa, synthetic construct
  REGION 1..128: note = SKA0046-VH
QEQLVESGGGLVQPEGSLTLTCTASGFSLSSSNYMWWVRQAPGKGLEWIACIHASSSGDTYYANWAKGRFTISKTSSTTVTLQMTSLTAADTATYFCARGGYIYGDGGYNGVAGFSLWGPGTLVTVSS

SEQ ID NO: 14 - protein, 123 aa, synthetic construct
  REGION 1..123: note = SKA0052-VH
QSLEESGGDLVKSGASLTLTCTASGFSFSSSYYMCWVRQAPGKGLEWIACIYTGSNGDTYYASWANGRFTISKASSTTVTLQMTSLTAADTATYFCARYTYDDDGDYTSFNLWGPGTLVTVSS

SEQ ID NO: 15 - protein, 127 aa, synthetic construct
  REGION 1..127: note = SKA0054-VH
QSLEESGGGLVQPEGSLTLTCTASGFSFSSSFWICWVRQAPGKGLEWLACIDAGSSDNTYYANWAKGRFTISKTSSTTVTLQMTSLTDADTATYFCARDRDAAGDAGYGYATSFNLWGPGTLVTVSS

SEQ ID NO: 16 - protein, 126 aa, synthetic construct
  REGION 1..126: note = SKA0117-VH
QSLEESGGDLVKPEGSLTLTCTASGFSFSSSHYMCWVRQAPGKGLEWIACIYAGSSDNTYYASWAKGRFTISKTSSTTVTLQMTSLTAADTATYFCARDTYGYVGYASAGGFFNLWGPGTLVTVSS

SEQ ID NO: 17 - protein, 114 aa, synthetic construct
  REGION 1..114: note = SKA0127-VH
QSVEESGGRLVTPGTPLTLTCTVSGIDLSTYAMTWVRQAPGKGLEYIGYINGGGSAYYASWVNGRFTISKTSTTADLKITSPTTEDTATYFCARGPVGSYHRIWGPGTLVTVSS

SEQ ID NO: 18 - protein, 123 aa, synthetic construct
  REGION 1..123: note = SKA0141-VH
QSLEESGGDLVKPGASLTLTCTASGFSFSSSYYMCWVRQAPGKGLEWIACIYAGSSGSTYYASWAKGRFTISKTSSTTVTLQMTSLTAADTATYFCARDGDENMLVGGYFNLWGPGTLVTVSS

SEQ ID NO: 19 - protein, 125 aa, synthetic construct
  REGION 1..125: note = SKA0171-VH
QSLEESGGGLVQPEGSLTLSCTASGFSFSSSYYMCWVRQAPGKGLEWIACIYGGNSVITYYANWAKGRFTISETSSTTVTLQMTSLTAADTATYFCAREVHYDYLYTTYGYANLWGPGTLVTVSS

SEQ ID NO: 20 - protein, 112 aa, synthetic construct
  REGION 1..112: note = SKA0001-VL
DIVMTQTPSSVSAAVGGTVTINCQASQSISRLLAWYQQKPGQPPKVLMHDASDLASGVPSRFKGSGSGTEYTLTISDLECADAATYYCQSYYWGSSGDIGYPFGGGTEVVVK

SEQ ID NO: 21 - protein, 110 aa, synthetic construct
  REGION 1..110: note = SKA0009-VL
AIEMTQTPSPVSAAVGGTVSINCQASEDIENYFAWYQQKPGQPPKLLIYDASELASGVPSRFSGSGSGTDFTLTISGVQCDDAATYYCQHADYAASSENTFGGGTEVVVK

SEQ ID NO: 22 - protein, 114 aa, synthetic construct
  REGION 1..114: note = SKA0016-VL
ADIVMTQTPSSVSAAVGGTVTIKCQASQNIYRNLAWYQQKPGQPPKLLIYQASKLESGVPSRFKGSGSGTEFTLTISDLDCDDAASYYCQSISYISSGDTFFWAFGGGTEVVVK

SEQ ID NO: 23 - protein, 110 aa, synthetic construct
  REGION 1..110: note = SKA0027-VL
AFELTQTPASVEAAVGGTVTIKCQASQNIYSNLAWYQQKPGQPPKLLIYTASSLASGVSSRFKGSGSGTEFTLTISDLECDDAATYYCQCTYINNGYGNTFGGGTEVVVK

SEQ ID NO: 24 - protein, 110 aa, synthetic construct
  REGION 1..110: note = SKA0028-VL
ALVMTQTPASVSEPVGGTVTINCQASQNIYSNLAWYQQKPGQRPRLLIYGASNLESGVPSRFRGSGSGTEFTLTISDLECADAATYYCQSAYWGNNNDGAFGGGTEVVVK

SEQ ID NO: 25 - protein, 113 aa, synthetic construct
  REGION 1..113: note = SKA0046-VL
DVVMTQTPASVSEPVGGTVTIKCQASETIGNALAWYQQKPGQRPKLLIYGASNLESGVPSRFKGSGSGTEFTLTISDVQCDDAATYYCQSIYYSSSADTFFFPFGGGTEVVVK

SEQ ID NO: 26 - protein, 113 aa, synthetic construct
  REGION 1..113: note = SKA0052-VL
ALVMTQTPSSVSADVGGTVTINCQASQNIYRNLAWYQQKPGQRPKLLIYGASNLEPGVPSRFKGSGSGTEFTLTISDLECDDAATYYCQSVGYSGSVDTFFYAFGGGTEVVVK

SEQ ID NO: 27 - protein, 113 aa, synthetic construct
  REGION 1..113: note = SKA0054-VL
AIDMTQTPSSVSAAVGGTVTINCQASESIYSNLAWYQQKPGQRPKLLIYLASTLASGVPSRFKGSGSGTEFTLTISDLECADAATYYCQSVYYDSRGDTFFWAFGGGTEVVVK

SEQ ID NO: 28 - protein, 111 aa, synthetic construct
  REGION 1..111: note = SKA0117-VL
ALVMTQTPASVSAAVGGTVTINCQASHNIYSVLAWYQQKPGQRPRLLIYGASNLESGVPSRFKGSGSGTEYTLTISDLECDDAATYYCQSAYYSSSTGDNAFGGGTEVVVK

SEQ ID NO: 29 - protein, 111 aa, synthetic construct
  REGION 1..111: note = SKA0127-VL
ALVMTQTPSSVSEPVGGTVTINCRASEDIESYLAWYQQKPGQPPKLLIYATSNLETGVPSRFKGSGSGTDYTLTISGVQCADAATYYCQGYYELSSIDNNAFGGGTEVVVK

SEQ ID NO: 30 - protein, 111 aa, synthetic construct
  REGION 1..111: note = SKA0141-VL
ALVMTQTPSSVSAAVGGTVTINCQASHNIYSNLAWYQQKPGQRPKLLIYGASNLESGVPSRFKGSGSGTEYTLTISDLECDDAATYYCQSAYYSSNAGYNAFGGGTEVVVK

SEQ ID NO: 31 - protein, 111 aa, synthetic construct
  REGION 1..111: note = SKA0171-VL
AIKMTQTPASVSAVVGGTVTINCQASETIYSGLAWYQQKPGQPPKLLIYDTANLETGVSSRFKGSGSGTEYTLTISDLECDDAATYFCLYAYYSGGSADNAFGGGTEVVVK

SEQ ID NO: 32 - protein, 5 aa, synthetic construct
  REGION 1..5: note = SKA0001-HCDR1
YYAMN

SEQ ID NO: 33 - protein, 5 aa, synthetic construct
  REGION 1..5: note = SKA0009-HCDR1
SYGMG

SEQ ID NO: 34 - protein, 6 aa, synthetic construct
  REGION 1..6: note = SKA0016-HCDR1
SSDYMC

SEQ ID NO: 35 - protein, 6 aa, synthetic construct
  REGION 1..6: note = SKA0027-HCDR1
SSDYMC

SEQ ID NO: 36 - protein, 5 aa, synthetic construct
  REGION 1..5: note = SKA0028-HCDR1
SYHMS

SEQ ID NO: 37 - protein, 6 aa, synthetic construct
  REGION 1..6: note = SKA0046-HCDR1
SSNYMW

SEQ ID NO: 38 - protein, 6 aa, synthetic construct
  REGION 1..6: note = SKA0052-HCDR1
SSYYMC

SEQ ID NO: 39 - protein, 6 aa, synthetic construct
  REGION 1..6: note = SKA0054-HCDR1
SSFWIC

SEQ ID NO: 40 - protein, 6 aa, synthetic construct
  REGION 1..6: note = SKA0117-HCDR1
SSHYMC

SEQ ID NO: 41 - protein, 5 aa, synthetic construct
  REGION 1..5: note = SKA0127-HCDR1
TYAMT

SEQ ID NO: 42 - protein, 6 aa, synthetic construct
  REGION 1..6: note = SKA0141-HCDR1
SSYYMC

SEQ ID NO: 43 - protein, 6 aa, synthetic construct
  REGION 1..6: note = SKA0171-HCDR1
SSYYMC

SEQ ID NO: 44 - protein, 16 aa, synthetic construct
  REGION 1..16: note = SKA0001-HCDR2
IIGISGGTYYASWAKG

SEQ ID NO: 45 - protein, 16 aa, synthetic construct
  REGION 1..16: note = SKA0009-HCDR2
IIYAAGRTYYASWVKG

SEQ ID NO: 46 - protein, 18 aa, synthetic construct
  REGION 1..18: note = SKA0016-HCDR2
CIYIGSNGDTFYASWAKG

SEQ ID NO: 47 - protein, 17 aa, synthetic construct
  REGION 1..17: note = SKA0027-HCDR2
CIYTGSGSTYYASWAKG

SEQ ID NO: 48 - protein, 16 aa, synthetic construct
  REGION 1..16: note = SKA0028-HCDR2
IIHGSGNMWYASWAKG

SEQ ID NO: 49 - protein, 18 aa, synthetic construct
  REGION 1..18: note = SKA0046-HCDR2
CIHASSSGDTYYANWAKG

SEQ ID NO: 50 - protein, 18 aa, synthetic construct
  REGION 1..18: note = SKA0052-HCDR2
CIYTGSNGDTYYASWANG

SEQ ID NO: 51 - protein, 18 aa, synthetic construct
  REGION 1..18: note = SKA0054-HCDR2
CIDAGSSDNTYYANWAKG

SEQ ID NO: 52 - protein, 18 aa, synthetic construct
  REGION 1..18: note = SKA0117-HCDR2
CIYAGSSDNTYYASWAKG

SEQ ID NO: 53 - protein, 16 aa, synthetic construct
  REGION 1..16: note = SKA0127-HCDR2
YINGGGSAYYASWVNG

SEQ ID NO: 54 - protein, 18 aa, synthetic construct
  REGION 1..18: note = SKA0141-HCDR2
CIYAGSSGSTYYASWAKG

SEQ ID NO: 55 - protein, 18 aa, synthetic construct
  REGION 1..18: note = SKA0171-HCDR2
CIYGGNSVITYYANWAKG

SEQ ID NO: 56 - protein, 8 aa, synthetic construct
  REGION 1..8: note = SKA0001-HCDR3
GGSAWGDL

SEQ ID NO: 57 - protein, 10 aa, synthetic construct
  REGION 1..10: note = SKA0009-HCDR3
HGSWYAGMDL

SEQ ID NO: 58 - protein, 14 aa, synthetic construct
  REGION 1..14: note = SKA0016-HCDR3
DSRRADDYGTRLDL

SEQ ID NO: 59 - protein, 12 aa, synthetic construct
  REGION 1..12: note = SKA0027-HCDR3
RGDSYSNYSFKL

SEQ ID NO: 60 - protein, 14 aa, synthetic construct
  REGION 1..14: note = SKA0028-HCDR3
IRFWASSNYYYFYL

SEQ ID NO: 61 - protein, 18 aa, synthetic construct
  REGION 1..18: note = SKA0046-HCDR3
GGYIYGDGGYNGVAGFSL

SEQ ID NO: 62 - protein, 14 aa, synthetic construct
  REGION 1..14: note = SKA0052-HCDR3
YTYDDDGDYTSFNL

SEQ ID NO: 63 - protein, 18 aa, synthetic construct
  REGION 1..18: note = SKA0054-HCDR3
DRDAAGDAGYGYATSFNL

SEQ ID NO: 64 - protein, 17 aa, synthetic construct
  REGION 1..17: note = SKA0117-HCDR3
DTYGYVGYASAGGFFNL

SEQ ID NO: 65 - protein, 9 aa, synthetic construct
  REGION 1..9: note = SKA0127-HCDR3
GPVGSYHRI

SEQ ID NO: 66 - protein, 14 aa, synthetic construct
  REGION 1..14: note = SKA0141-HCDR3
DGDENMLVGGYFNL

SEQ ID NO: 67 - protein, 16 aa, synthetic construct
  REGION 1..16: note = SKA0171-HCDR3
EVHYDYLYTTYGYANL

SEQ ID NO: 68 - protein, 11 aa, synthetic construct
  REGION 1..11: note = SKA0001-LCDR1
QASQSISRLLA

SEQ ID NO: 69 - protein, 11 aa, synthetic construct
  REGION 1..11: note = SKA0009-LCDR1
QASEDIENYFA

SEQ ID NO: 70 - protein, 11 aa, synthetic construct
  REGION 1..11: note = SKA0016-LCDR1
QASQNIYRNLA

SEQ ID NO: 71 - protein, 11 aa, synthetic construct
  REGION 1..11: note = SKA0027-LCDR1
QASQNIYSNLA

SEQ ID NO: 72 - protein, 11 aa, synthetic construct
  REGION 1..11: note = SKA0028-LCDR1
QASQNIYSNLA

SEQ ID NO: 73 - protein, 11 aa, synthetic construct
  REGION 1..11: note = SKA0046-LCDR1
QASETIGNALA

SEQ ID NO: 74 - protein, 11 aa, synthetic construct
  REGION 1..11: note = SKA0052-LCDR1
QASQNIYRNLA

SEQ ID NO: 75 - protein, 11 aa, synthetic construct
  REGION 1..11: note = SKA0054-LCDR1
QASESIYSNLA

SEQ ID NO: 76 - protein, 11 aa, synthetic construct
  REGION 1..11: note = SKA0117-LCDR1
QASHNIYSVLA

SEQ ID NO: 77 - protein, 11 aa, synthetic construct
  REGION 1..11: note = SKA0127-LCDR1
RASEDIESYLA

SEQ ID NO: 78 - protein, 11 aa, synthetic construct
  REGION 1..11: note = SKA0141-LCDR1
QASHNIYSNLA

SEQ ID NO: 79 - protein, 11 aa, synthetic construct
  REGION 1..11: note = SKA0171-LCDR1
QASETIYSGLA

SEQ ID NO: 80 - protein, 7 aa, synthetic construct
  REGION 1..7: note = SKA0001-LCDR2
DASDLAS

SEQ ID NO: 81 - protein, 7 aa, synthetic construct
  REGION 1..7: note = SKA0009-LCDR2
DASELAS

SEQ ID NO: 82 - protein, 7 aa, synthetic construct
  REGION 1..7: note = SKA0016-LCDR2
QASKLES

SEQ ID NO: 83 - protein, 7 aa, synthetic construct
  REGION 1..7: note = SKA0027-LCDR2
TASSLAS

SEQ ID NO: 84 - protein, 7 aa, synthetic construct
  REGION 1..7: note = SKA0028-LCDR2
GASNLES

SEQ ID NO: 85 - protein, 7 aa, synthetic construct
  REGION 1..7: note = SKA0046-LCDR2
GASNLES

SEQ ID NO: 86 - protein, 7 aa, synthetic construct
  REGION 1..7: note = SKA0052-LCDR2
GASNLEP

SEQ ID NO: 87 - protein, 7 aa, synthetic construct
  REGION 1..7: note = SKA0054-LCDR2
LASTLAS

SEQ ID NO: 88 - protein, 7 aa, synthetic construct
  REGION 1..7: note = SKA0117-LCDR2
GASNLES

SEQ ID NO: 89 - protein, 7 aa, synthetic construct
  REGION 1..7: note = SKA0127-LCDR2
ATSNLET

SEQ ID NO: 90 - protein, 7 aa, synthetic construct
  REGION 1..7: note = SKA0141-LCDR2
GASNLES

SEQ ID NO: 91 - protein, 7 aa, synthetic construct
  REGION 1..7: note = SKA0171-LCDR2
DTANLET

SEQ ID NO: 92 - protein, 14 aa, synthetic construct
  REGION 1..14: note = SKA0001-LCDR3
QSYYWGSSGDIGYP

SEQ ID NO: 93 - protein, 12 aa, synthetic construct
  REGION 1..12: note = SKA0009-LCDR3
QHADYAASSENT

SEQ ID NO: 94 - protein, 15 aa, synthetic construct
  REGION 1..15: note = SKA0016-LCDR3
QSISYISSGDTFFWA

SEQ ID NO: 95 - protein, 12 aa, synthetic construct
  REGION 1..12: note = SKA0027-LCDR3
QCTYINNGYGNT

SEQ ID NO: 96 - protein, 12 aa, synthetic construct
  REGION 1..12: note = SKA0028-LCDR3
QSAYWGNNNDGA

SEQ ID NO: 97 - protein, 15 aa, synthetic construct
  REGION 1..15: note = SKA0046-LCDR3
QSIYYSSSADTFFFP

SEQ ID NO: 98 - protein, 15 aa, synthetic construct
  REGION 1..15: note = SKA0052-LCDR3
QSVGYSGSVDTFFYA

SEQ ID NO: 99 - protein, 15 aa, synthetic construct
  REGION 1..15: note = SKA0054-LCDR3
QSVYYDSRGDTFFWA

SEQ ID NO: 100 - protein, 13 aa, synthetic construct
  REGION 1..13: note = SKA0117-LCDR3
QSAYYSSSTGDNA

SEQ ID NO: 101 - protein, 13 aa, synthetic construct
  REGION 1..13: note = SKA0127-LCDR3
QGYYELSSIDNNA

SEQ ID NO: 102 - protein, 13 aa, synthetic construct
  REGION 1..13: note = SKA0141-LCDR3
QSAYYSSNAGYNA

SEQ ID NO: 103 - protein, 13 aa, synthetic construct
  REGION 1..13: note = SKA0171-LCDR3
LYAYYSGGSADNA

SEQ ID NO: 104 - protein, 5 aa, synthetic construct
  REGION 1..5: note = g-silent
RRGPK

SEQ ID NO: 105 - protein, 13 aa, synthetic construct
  REGION 1..13: note = ACT-5
LHEALHAHYTRKE

SEQ ID NO: 106 - protein, 330 aa, Homo sapiens [sequence omitted]

SEQ ID NO: 107 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 108 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 109 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 328 aa, synthetic construct
  REGION 1..328: note = Anti-IL-8 Antibody_H_C
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 111 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 112 - protein, 116 aa, synthetic construct
  REGION 1..116: note = Mouse anti-IL-8 mAb_VH
QVQLQQPGVELVMPGASVKLSCKASGYTFTSYWMHWVKQRPGQGLEWIGEIDPSDSNTNYNQKFKGKATLTLDKSSSTAYMQLTSLTSEDSAVYYCARELLHAVYWGQGTLVTVSS

SEQ ID NO: 113 - protein, 106 aa, synthetic construct
  REGION 1..106: note = Mouse anti-IL-8 mAb_VL
DIQMTQSPSSLSASLGGKVTITCTASQDIHKYISWFQHKPGKGPRLLIHYTSTLQPGIPSRFSGSGSGRDYSFSISNLEPEDVATYYCLQYDNLWTFGGGTKLDIK

SEQ ID NO: 114 - protein, 330 aa, synthetic construct
  REGION 1..330: note = Mouse anti-IL-8 mAb_CH
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 115 - protein, 107 aa, synthetic construct
  REGION 1..107: note = Mouse anti-IL-8 mAb_CL
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 116 - protein, 328 aa, synthetic construct
  REGION 1..328: note = SG1106
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 117 - protein, 5 aa, synthetic construct
  REGION 1..5: note = g-silent_IL-8
RRGPS

SEQ ID NO: 118 - protein, 13 aa, synthetic construct
  REGION 1..13: note = ACT-5_IL-8
LHEALHAHTTRKE